(19) United States
(12) Patent Application Publication
(10) Pub. No.: US 2010/0234435 A1
(43) Pub. Date: Sep. 16, 2010

(54) CYCLOALKYLAMINO ACID DERIVATIVES

(76) Inventors: Samit K. Bhattacharya, Niantic, CT (US); Matthew F. Brown, Stonington, CT (US); Peter H. Dorff, Griswold, CT (US); Susan D. LaGreca, Old Lyme, CT (US); John W. Mickelson, Lakeville, MN (US); Joseph A. Cornicelli, Ann Arbor, MI (US); David L. Brown, Chesterfield, MO (US); Jennings Rex, Voluntown, CT (US); John K. Walker, Weldon Springs, MO (US); Rita Huff, Wildwood, MO (US); Joseph W. Strohbach, Wentzville, MO (US); Robert J. Maguire, East Lyme, CT (US)

Correspondence Address: PHARMACIA CORPORATION;c/o Pfizer Inc., Eastern Point Road, MS9114, Groton, CT, 06340, US

(21) Appl. No.: 12/779,379

(22) Filed: May 13, 2010

Related U.S. Application Data
(63) Continuation of application No. 12/266,997, filed on Nov. 7, 2008.
(60) Provisional application No. 60/986,480, filed on Nov. 8, 2007.

(51) Int. Cl. A61K 31/4245; C07D 271/06; A61P 19/02; C07D 405/14; C07D 401/04

(52) U.S. Cl. 514/364; 548/131; 544/333; 546/268.4

(57) ABSTRACT

The invention relates to compounds of formula I

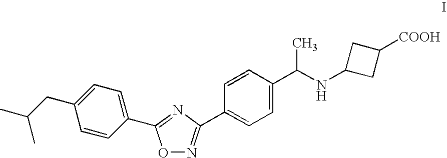

and to pharmaceutically acceptable salts, prodrugs, solvates or hydrates thereof. This invention also relates to a method of using such compounds in the treatment of hyperproliferative diseases and autoimmune diseases in mammals, especially humans, and to pharmaceutical compositions containing such compounds.

### BACKGROUND OF THE INVENTION

This invention relates to novel carboxycycloalkylamino derivatives. The carboxycycloalkylamino derivatives of the present invention are modulators of the sphingosine-1-phosphate (S1P) receptors and have a number of therapeutic applications, particularly in the treatment of hyperproliferative, inflammatory diseases including atherosclerosis and liver fibrosis, and autoimmune diseases, in mammals, especially humans, and to pharmaceutical compositions containing such compounds.

The S1P receptors 1-5 constitute a family of seven-transmembrane G-protein coupled receptors. These receptors, referred to as S1P1 to S1P5, are activated via binding by sphingosine-1-phosphate, which is produced by the sphingosine kinase phosphorylation of sphingosine. S1P receptors are cell surface receptors involved in a variety of cellular processes, including cell proliferation and differentiation, cell survival, and cell migration. S1P is found in plasma and a variety of other tissues and exerts autocrine and paracrine effects.

Recent studies indicate that S1P binds to the S1P1 receptor to promote tumor angiogenesis by supporting the migration, proliferation and survival of endothelial cells (ECs) as they form new vessels within tumors (tumor angiogenesis) (Lee et al., Cell. 99:301-312 (1999) Paik et al., J. Biol. Chem. 276:11830-11837 (2001)). Because S1P is required for optimal activity of multiple proangiogenic factors, modulating S1P1 activation may affect angiogenesis, proliferation, and interfere with tumor neovascularization, vessel maintenance and vascular permeability.

Other diseases or conditions that may be treated with the compounds of the present invention include organ transplant rejection and inflammatory diseases, which are believed to proceed via modulating the S1P receptors.

Thus, the identification of compounds which modulate the activity of the S1P1 receptor to regulate and modulate abnormal or inappropriate cell proliferation, differentiation, or metabolism is therefore desirable.

Patent application U.S. Ser. No. 11/746,314, filed May 9, 2007 (claiming the benefit of priority to U.S. provisional application Ser. No. 60/799,210, filed May 9, 2006), describes S₁P₁ inhibitors of formula:

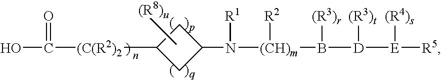

or the pharmaceutically acceptable salts thereof;

wherein B is selected from the group consisting of phenyl and a (5 to 6-membered)-heteroaryl ring;

D is selected from the group consisting of phenyl and a (5 to 6-membered)-heteroaryl ring;

E is selected from the group consisting of phenyl and a (5 to 6-membered)-heteroaryl ring;

R¹ is a radical selected from the group consisting of hydrogen, (C₁-C₆)alkyl-, (C₂-C₆)alkenyl-, (C₂-C₆)alkynyl-, (C₃-C₇)cycloalkyl-, (C₂-C₉)heterocyclyl-, (C₆-C₁₀)aryl-, (C₁-C₁₂)heteroaryl-, R⁷—SO₂—, R⁷—C(O)—, R⁷O—C(O)—, and (R⁷)₂N—C(O)—;

wherein each of said (C₁-C₆)alkyl-, (C₂-C₆)alkenyl-, (C₂-C₆)alkynyl-, (C₃-C₇)cycloalkyl-, (C₂-C₉)heterocyclyl-, (C₆-C₁₀)aryl-, (C₁-C₁₂)heteroaryl-, R⁷—SO₂—, R⁷—C(O)—, R⁷O—C(O)—, and (R⁷)₂N—C(O)—R¹ radicals may optionally be substituted by one to three moieties independently selected from the group consisting of hydrogen, hydroxy, halogen, —CN, (C₁-C₆)alkyl-, (C₁-C₆)alkoxy-, perhalo(C₁-C₆)alkyl-, (C₃-C₇)cycloalkyl-, (C₂-C₉)heterocyclyl-, (C₆-C₁₀)aryl-, and (C₁-C₁₂)heteroaryl-;

each R² is a radical independently selected from the group consisting of hydrogen, hydroxy, halogen, —CN, (C₁-C₆)alkyl-, (C₂-C₆)alkenyl-, (C₂-C₆)alkynyl-, (C₃-C₇)cycloalkyl-, (C₂-C₉)heterocyclyl-, (C₆-C₁₀)aryl-, and (C₁-C₁₂)heteroaryl-;

wherein each of said (C₁-C₆)alkyl-, (C₂-C₆)alkenyl-, (C₂-C₆)alkynyl-, (C₃-C₇)cycloalkyl-, (C₂-C₉)heterocyclyl-, (C₆-C₁₀)aryl-, and (C₁-C₁₂)heteroaryl-R² radicals may optionally be substituted by one to three moieties independently selected from the group consisting of hydrogen, hydroxy, halogen, —CN, (C₁-C₆)alkyl-, perhalo(C₁-C₄)alkyl-, perhalo(C₁-C₄)alkoxy-, (C₃-C₇)cycloalkyl-, (C₂-C₉)heterocyclyl-, (C₆-C₁₀)aryl-, and (C₁-C₁₂)heteroaryl-;

each R³ is a radical independently selected from the group consisting of hydrogen, halogen, hydroxy, —CN, (C₁-C₆)alkyl-, (C₂-C₆)alkenyl-, (C₂-C₆)alkynyl-, (C₃-C₇)cycloalkyl-, (C₁-C₆)alkoxy-, perhalo(C₁-C₆)alkyl-, and perhalo(C₁-C₆)alkoxy-;

each R⁴ is a radical independently selected from the group consisting of hydrogen, halogen, hydroxy, —CN, —N(R⁸)₂, (C₁-C₆)alkyl-, (C₂-C₆)alkenyl-, (C₃-C₆)alkynyl-, (C₁-C₆)alkoxy-, perhalo(C₁-C₆)alkyl-, (C₁-C₆)alkyl-S(O)ₖ—, R¹⁰C(O)N(R¹⁰)—, (R¹⁰)₂NC(O)—, R¹⁰C(O)—, R¹⁰OC(O)—, (R¹⁰)₂NC(O)N(R¹⁰)—, (R¹⁰)₂NS(O)—, (R¹⁰)₂NS(O)₂—, (C₃-C₇)cycloalkyl-, (C₆-C₁₀)aryl-, (C₂-C₉)heterocyclyl-, and (C₁-C₁₂)heteroaryl-;

wherein each of said (C₁-C₆)alkyl-, (C₂-C₆)alkenyl-, (C₃-C₆)alkynyl-, (C₁-C₆)alkoxy-(C₁-C₆)-alkyl-S(O)ₖ—, R¹⁰C(O)N(R¹⁰)—, (R¹⁰)₂NC(O)—, R¹⁰C(O)—, R¹⁰OC(O)—, (R¹⁰)₂NC(O)N(R¹⁰)—, (R¹⁰)₂NS(O)—, (R¹⁰)₂NS(O)₂—, (C₃-C₇)cycloalkyl-, (C₆-C₁₀)aryl-, (C₂-C₉)heterocyclyl-, and (C₁-C₁₂)heteroaryl-R⁴ radicals may optionally be substituted from one to five moieties independently selected from the group consisting of halogen, hydroxy, —CN, (C₁-C₆)alkyl-, (C₃-C₇)cycloalkyl, —(C₁-C₆)alkoxy and -perhalo(C₁-C₆)alkoxy;

R⁵ is a radical selected from the group consisting of hydrogen, halogen, —CN, (C₁-C₁₀)alkyl-, (C₁-C₆)alkoxy-, (C₂-C₁₀)alkenyl-, (C₂-C₁₀alkynyl-, (C₃-C₇)cycloalkyl-, (C₆-C₁₀)aryl-, (C₂-C₉)heterocyclyl-, (C₁-C₁₂)heteroaryl-, (C₃-C₇)cycloalkyl-O—, (C₆-C₁₀)aryl-O—, (C₂-C₉)heterocyclyl-O—, (C₁-C₁₂)heteroaryl-O—, R⁷—S—, R⁷—SO—, R⁷—SO₂—, R⁷—C(O)—, R⁷—C(O)—O—, R⁷O—C(O)—, and (R⁷)₂N—C(O)—;

wherein each of said (C₁-C₁₀)alkyl-, (C₁-C₆)alkoxy- and (C₂-C₁₀)alkynyl-R⁵ radicals may optionally be substituted with from one to five moieties independently selected from the group consisting of halogen, hydroxy, —CN, (C₁-C₆)alkyl-, (C₃-C₇)cycloalkyl-, (C₆-C₁₀)aryl-, (C₁-C₆)alkoxy-, (C₂-C₉)heterocyclyl-, and (C₁-C₁₂)heteroaryl-;

wherein each of said (C₃-C₇)cycloalkyl- and (C₃-C₇)cycloalkyl-O—R⁵ radicals may optionally be substituted with from one to five moieties independently selected from the group consisting of halogen, hydroxy, —CN, (C₁-C₆)alkyl-, (C₆-C₁₀)aryl-, (C₁-C₆)alkoxy-, (C₂-C₉)heterocyclyl-, and (C₁-C₁₂)heteroaryl-;

wherein each of said (C₆-C₁₀)aryl-, (C₂-C₉)heterocyclyl-, (C₁-C₁₂)heteroaryl-, (C₆-C₁₀)aryl-O—, (C₂-C₉)heterocyclyl-O—, and (C₁-C₁₂)heteroaryl-O—R⁵ radicals may optionally be substituted with from one to five moieties independently selected from the group consisting of halogen, hydroxy, —CN, (C₁-C₆)alkyl-, and (C₁-C₆)alkoxy-;

wherein each of said R⁷—S—, R⁷—SO—, R⁷—SO₂—, R⁷—C(O)—, R⁷—C(O)—O—, R⁷O—C(O)—, and (R⁷)₂N—C(O)—R⁵ radicals may optionally be substituted with from one to five moieties independently selected from the group consisting of halogen, hydroxy, —CN, (C₁-C₆)alkyl-, (C₃-C₇)cycloalkyl, and (C₁-C₆)alkoxy-;

wherein each of aforesaid (C₁-C₆)alkyl-, (C₁-C₆)alkoxy-, (C₆-C₁₀)aryl-, (C₁-C₆)alkoxy-, (C₂-C₉)heterocyclyl-, and (C₁-C₁₂)heteroaryl-moieties for each of aforesaid R⁵ radicals may optionally be substituted with one to five halogen groups;

optionally said R⁵ radical and one R⁴ radical or two R⁴ radicals may be taken together with E to form an (8 to 10-membered)-fused bicyclic ring optionally containing 1 to 4 heteroatoms selected from the group consisting of O, S, or N(R⁶);

wherein said (8 to 10-membered)-fused bicyclic ring is additionally optionally substituted with one to two oxo (═O) groups;

each R⁶ is a bond or a radical independently selected from the group consisting of hydrogen, (C₁-C₆)alkyl-, —CN, and perhalo(C₁-C₆)alkyl-;

each R⁷ is a radical independently selected from the group consisting of hydrogen, —CN, (C₁-C₆)alkyl-, perhalo(C₁-C₆)alkyl-, (C₂-C₆)alkenyl-, (C₂-C₆)alkynyl-, (C₃-C₇)cycloalkyl-, (C₂-C₉)heterocyclyl-, (C₆-C₁₀)aryl-, and (C₁-C₁₂)heteroaryl-;

each R⁸ is a radical independently selected from the group consisting of hydrogen, hydroxy, halogen, —CN, —NH(R⁹), (C₁-C₆)alkyl-, perhalo(C₁-C₆)alkyl- and (C₁-C₆)alkoxy-;

wherein each of said (C₁-C₆)alkyl- and (C₁-C₆)alkoxy-R⁸ radicals is optionally substituted from one to five moieties selected from the group consisting of perhalo(C₁-C₆)alkyl-, —O(R⁹) and —N(R⁹)₂;

each R⁹ is a radical independently selected from the group consisting of hydrogen, (C₁-C₆)alkyl-, (C₂-C₆)alkenyl-, (C₂-C₆)alkynyl-, (C₃-C₇)cycloalkyl-, (C₂-C₉)heterocyclyl-, (C₆-C₁₀)aryl-, (C₁-C₁₂)heteroaryl-, R⁷—SO₂—, R⁷—C(O)—, R⁷—C(O)—O—, R⁷O—C(O)—, and (R⁷)₂N—C(O)—;

wherein each of said (C₁-C₆)alkyl-, (C₂-C₆)alkenyl-, (C₂-C₆)alkynyl-, (C₃-C₇)cycloalkyl-, (C₂-C₉)heterocyclyl-, (C₆-C₁₀)aryl-, (C₁-C₁₂)heteroaryl-R⁹ radicals is optionally substituted by one to three moieties independently selected from the group consisting of hydrogen, hydroxy, halogen, —CN, (C₁-C₆)alkyl-, (C₁-C₆)alkoxy-, perhalo(Cᵣ C₆)alkyl-, (C₃-C₇)cycloalkyl-, (C₂-C₉)heterocyclyl-, (C₆-C₁₀)aryl-, and (C₁-C₁₂)heteroaryl-;

each R¹⁰ is a radical selected from the group consisting of hydrogen and (C₁-C₆)alkyl-;

k is an integer from 0 to 2;

m and n are each independently an integer from 0 to 3;

p is an integer from 1 to 2;

q is an integer from 0 to 2; and

r, s, t and u are each independently an integer from 0 to 4.

### SUMMARY OF THE INVENTION

There have now been found compounds that exhibit lower clearance, and thus longer half-lives, as compared with what is believed to be the closest compound exemplified in U.S. Ser. No. 11/746,314, page 16, lines 21-22 and page 110, Example 34B, 3-{3-[5-(4-Isobutyl-phenyl)-[1,2,4]oxadiazole-3-yl]-benzylamino}-cis-cyclobutanecarboxylic acid, having the structure:

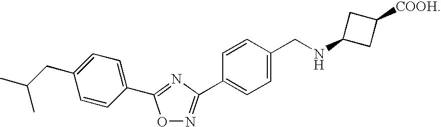

In addition, the compounds of the present invention exhibit higher selectivity over S1P₄, as compared with 3-{3-[5-(4-Isobutyl-phenyl)-[1,2,4]oxadiazole-3-yl]-benzylamino}-cis-cyclobutanecarboxylic acid.

The present invention related to a compound of the formula I

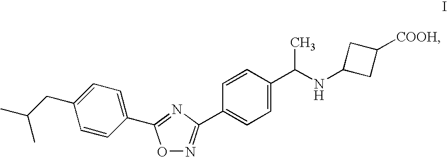

or the pharmaceutically acceptable salts thereof.

Formula I contains one chiral center and one cis/trans isomeric center meaning compounds of formula I can exist as 4 possible stereoisomers. The following structures are individually included as compounds of the present invention:

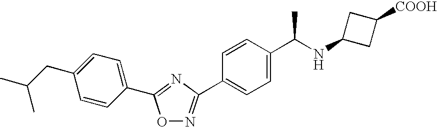

cis-3-((R)-1-(4-(5-(4-isobutylphenyl)-1,2,4-oxadiazol-3-yl)phenyl)ethylamino)cyclobutanecarboxylic acid;

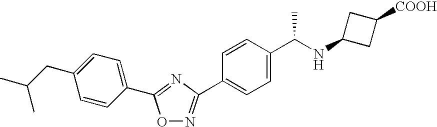

cis-3((S)-1-(4-(5-(4-isobutylphenyl)-1,2,4-oxadiazol-3-yl)phenyl)ethylamino)cyclobutanecarboxylic acid;

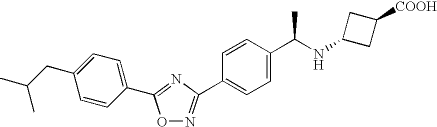

trans-3-((R)-1-(4-(5-(4-isobutylphenyl)-1,2,4-oxadiazol-3-yl)phenyl)ethylamino)cyclobutanecarboxylic acid; and

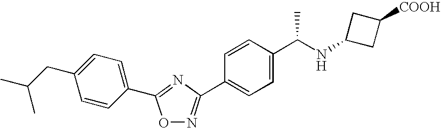

trans-3-((S)-1-(4-(5-(4-isobutylphenyl)-1,2,4-oxadiazol-3-yl)phenyl)ethylamino)cyclobutanecarboxylic acid.

As used herein, the phrase “compound of formula I” and “pharmaceutically acceptable salts” includes prodrugs, metabolites, solvates or hydrates thereof.

More specifically, the present invention includes pharmaceutically acceptable acid addition salts of compounds of the formula I. The acids which are used to prepare the pharmaceutically acceptable acid addition salts of the aforementioned base compounds of this invention are those which form non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, naphthalate, p-toluenesulfonate and pamoate [i.e. 1,1′-methylene-bis-(2-hydroxy-3-naphthoate)] salts.

The invention also includes base addition salts of formula I. The chemical bases that may be used as reagents to prepare pharmaceutically acceptable base salts of those compounds of formula I that are acidic in nature are those that form non-toxic base salts with such compounds. Such non-toxic base salts include, but are not limited to those derived from such pharmacologically acceptable cations such as alkali metal cations (e.g., potassium and sodium) and alkaline earth metal cations (e.g., calcium and magnesium), ammonium or water-soluble amine addition salts such as N-methylglucamine-(meglumine), and the lower alkanolammonium and other base salts of pharmaceutically acceptable organic amines.

Hemisalts of acids and bases may also be formed, for example, hemisulphate and hemicalcium salts.

For a review on suitable salts, see Handbook of Pharmaceutical Salts: Properties, Selection, and Use by Stahl and Wermuth (Wiley-VCH, 2002).

As indicated, so-called ‘prodrugs’ of the compounds of formula I are also within the scope of the invention. Thus certain derivatives of compounds of formula I which may have little or no pharmacological activity themselves can, when administered into or onto the body, be converted into compounds of formula I having the desired activity, for example, by hydrolytic cleavage. Such derivatives are referred to as ‘prodrugs’. Further information on the use of prodrugs may be found in Pro-drugs as Novel Delivery Systems, Vol. 14, ACS Symposium Series (T. Higuchi and W. Stella) and Bioreversible Carriers in Drug Design, Pergamon Press, 1987 (Ed. E. B. Roche, American Pharmaceutical Association).

Prodrugs in accordance with the invention can, for example, be produced by replacing appropriate functionalities present in the compounds of formula I with certain moieties known to those skilled in the art as ‘pro-moieties’ as described, for example, in Design of Prodrugs by H. Bundgaard (Elsevier, 1985).

Some examples of prodrugs in accordance with the invention include

(i) since the compound of formula I contains a carboxylic acid functionality (—COOH), an ester thereof, for example, a compound wherein the hydrogen of the carboxylic acid functionality of the compound of formula I is replaced by (C₁-C₈)alkyl; and

(ii) since the compound of formula I contains a secondary amino functionality (—NHR where R≠H), an amide thereof, for example, a compound wherein, as the case may be, one or both hydrogens of the amino functionality of the compound of formula I is/are replaced by (C₁-C₁₀)alkanoyl.

Further examples of replacement groups in accordance with the foregoing examples and examples of other prodrug types may be found in the aforementioned references.

Also included within the scope of the invention are metabolites of compounds of formula I, that is, compounds formed in vivo upon administration of the drug. Some examples of metabolites in accordance with the invention include

(i) since the compound of formula I contains a methyl group, an hydroxymethyl derivative thereof (—CH₃->—CH₂OH):

(ii) since the compound of formula I contains a secondary amino group, a primary derivative thereof (—NHR¹->—NH₂); and

(iii) since the compound of formula I contains a phenyl moiety, a phenol derivative thereof (-Ph->-PhOH).

Included within the scope of the present invention are all stereoisomers of the compounds of formula I, including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Each of the aforesaid species of the invention includes the pharmaceutically acceptable salts, prodrugs, hydrates or solvates of the aforementioned compound.

In one embodiment of the present invention, a method of treating inflammation or an inflammation-related disorder or an autoimmune diseases is provided.

For example, compounds of the present invention will be useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis.

Compounds of the invention will be further useful in the treatment of asthma, bronchitis, menstrual cramps (e.g., dysmenorrhea), premature labor, tendinitis, bursitis, skin-related conditions such as psoriasis, eczema, burns, sunburn, dermatitis, pancreatitis, hepatitis, and post-operative inflammation including inflammation from ophthalmic surgery such as cataract surgery and refractive surgery. Compounds of the invention also would be useful to treat gastrointestinal conditions such as inflammatory bowel disease, Crohn's disease, gastritis, irritable bowel syndrome, ulcerative colitis. Celiac disease, alopecia areata, myloidosis. Hashimoto's disease, juvenile diabetes. Lyme disease, peripheral neuropathy, and vasculitis.

Compounds of the invention would be useful in treating inflammation and tissue damage in such diseases as vascular diseases, migraine headaches, periarteritis nodosa, thyroiditis, aplastic anemia. Hodgkin's disease, sclerodoma, rheumatic fever, type I diabetes, neuromuscular junction disease including myasthenia gravis, white matter disease including multiple sclerosis, sarcoidosis, nephrotic syndrome. Behcet's syndrome, polymyositis, gingivitis, nephritis, hypersensitivity, swelling occurring after injury, myocardial ischemia, and the like. The compounds would also be useful in the treatment of ophthalmic diseases, such as glaucoma, retinitis, retinopathies, uveitis, ocular photophobia, and of inflammation and pain associated with acute injury to the eye tissue. The compounds would also be useful in the treatment of pulmonary inflammation, such as that associated with viral infections and cystic fibrosis. The compounds would also be useful for the treatment of certain central nervous system disorders, such as cortical dementias including Alzheimer's disease, Guillain-Barre syndrome and central nervous system damage resulting from stroke, ischemia and trauma. These compounds would also be useful in the treatment of allergic rhinitis, respiratory distress syndrome, endotoxin shock syndrome, and atherosclerosis. The compounds would also be useful in the treatment of pain, including but not limited to postoperative pain, dental pain, muscular pain, neuropathic pain, pain caused by temperoramandibular joint syndrome, and pain resulting from cancer.

Besides being useful for human treatment, these compounds are also useful for veterinary treatment of companion animals, exotic animals and farm animals, including mammals and other vertebrates. More preferred animals include horses, dogs, and cats.

This invention also relates to a method for the treatment of abnormal cell growth in a mammal, preferably a human, comprising administering to said mammal an amount of a compound of the Formula I, or a pharmaceutically acceptable salt thereof (including hydrates, solvates and polymorphs of said compound of Formula I or pharmaceutically acceptable salts thereof), that is effective in treating abnormal cell growth.

In one embodiment of this method, the abnormal cell growth is cancer, including, but not limited to, mesothelioma, hepatobilliary cancers (hepatic and biliary duct), a primary or secondary CNS tumor, a primary or secondary brain tumor (including pituitary tumors, astrocytomas, meningiomas and medulloblastomas), lung cancer (NSCLC and SCLC), bone cancer, pancreatic cancer, skin cancer, cancer of the head or neck, cutaneous or intraocular melanoma, ovarian cancer, colon cancer, rectal cancer, liver cancer, cancer of the anal region, stomach cancer, gastrointestinal (gastric, colorectal, and duodenal), breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, gastrointestinal stromal tumor (GIST), pancreatic endocrine tumors (such as pheochromocytoma, insulinoma, vasoactive intestinal peptide tumor, islet cell tumor and glucagonoma), carcinoid tumors, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, tumors of the blood vessels (including benign and malignant tumors such as hemangiomas, hemangiosarcomas, hemangioblastomas and lobular capillary hemangiomas) or a combination of one or more of the foregoing cancers.

Another more specific embodiment of the present invention is directed to a cancer selected from lung cancer (NSCLC and SCLC), cancer of the head or neck, ovarian cancer, colon cancer, rectal cancer, cancer of the anal region, stomach cancer, breast cancer, cancer of the kidney or ureter, renal cell carcinoma, carcinoma of the renal pelvis, neoplasms of the central nervous system (CNS), primary CNS lymphoma, non-Hodgkins's lymphoma, spinal axis tumors, or a combination of one or more of the foregoing cancers.

In another more specific embodiment of the present invention the cancer is selected from lung cancer (NSCLC and SCLC), breast cancer, ovarian cancer, colon cancer, rectal cancer, cancer of the anal region, or a combination of one or more of the foregoing cancers.

In another embodiment of the present invention, said abnormal cell growth is a benign proliferative disease, including, but not limited to, psoriasis, benign prostatic hypertrophy, restinosis, synovial proliferation disorder, retinopathy or other neovascular disorders of the eye, pulmonary hypertension from bone marrow for use in reconstituting normal cells of any tissue.

This invention also relates to a method for the treatment of abnormal cell growth in a mammal in need of such treatment, which comprises administering to said mammal an amount of a compound of Formula I (including hydrates, solvates and polymorphs of said compound of formula I or pharmaceutically acceptable salts thereof), in combination with one or more (preferable one to three) anti-cancer agents selected from the group consisting of traditional anticancer agents (such as DNA binding agents, mitotic inhibitors, alkylating agents, anti-metabolites, intercalating antibiotics, topoisomerase inhibitors and microtubulin inhibitors), statins, radiation, angiogenesis inhibitors, signal transduction inhibitors, cell cycle inhibitors, telomerase inhibitors, biological response modifiers (such as antibodies, immunotherapy and peptide mimics), anti-hormones, anti-androgens, gene silencing agents, gene activating agents and anti-vascular agents, wherein the amounts of the compound of Formula I together with the amounts of the combination anticancer agents is effective in treating abnormal cell growth.

The invention also relates to a method for the treatment of a hyperproliferative disorder in a mammal in need of such treatment, comprising administering to said mammal an amount of a compound of Formula I (including hydrates, solvates and polymorphs of said compound of Formula I or pharmaceutically acceptable salts thereof), in combination with an anti-cancer agent selected from the group consisting of traditional anticancer agents (such as DNA binding agents, mitotic inhibitors, alkylating agents, anti-metabolites, intercalating antibiotics, topoisomerase inhibitors and microtubulin inhibitors), statins, radiation, angiogenesis inhibitors, signal transduction inhibitors, cell cycle inhibitors, telomerase inhibitors, biological response modifiers (such as antibodies, immunotherapy and peptide mimics), hormones, anti-hormones, anti-androgens, gene silencing agents, gene activating agents and anti-vascular agents, wherein the amounts of the compound of Formula I together with the amounts of the combination anticancer agents is effective in treating said hyperproliferative disorder.

This invention also relates to a pharmaceutical composition comprising an amount of a compound of the Formula I, as defined above (including hydrates, solvates and polymorphs of said compound of Formula I or pharmaceutically acceptable salts thereof), and a pharmaceutically acceptable carrier.

The invention also relates to a pharmaceutical composition which comprises an amount of a compound of Formula I, as defined above (including hydrates, solvates and polymorphs of said compound of formula I or pharmaceutically acceptable salts thereof), in combination with one or more (preferably one to three) anti-cancer agent selected from the group consisting of traditional anticancer agents (such as DNA binding agents, mitotic inhibitors, alkylating agents, anti-metabolites, intercalating antibiotics, topoisomerase inhibitors and microtubulin inhibitors), statins, radiation, angiogenesis inhibitors, signal transduction inhibitors, cell cycle inhibitors, telomerase inhibitors, biological response modifiers, hormones, anti-hormones, anti-androgens gene silencing agents, gene activating agents and anti-vascular agents and a pharmaceutically acceptable carrier, wherein the amounts of the compound of Formula I and the combination anti-cancer agents when taken as a whole is therapeutically effective for treating said abnormal cell growth.

In one embodiment of the present invention the anti-cancer agent used in conjunction with a compound of Formula I and pharmaceutical compositions described herein is an anti-angiogenesis agent.

A more specific embodiment of the present invention includes combinations of the compounds of Formula I with anti-angiogenesis agents selected from VEGF inhibitors, VEGFR inhibitors, TIE-2 inhibitors, PDGFR inhibitors, angiopoetin inhibitors. PKCβ inhibitors, COX-2 (cyclooxygenase II) inhibitors, integrins (alpha-v/beta-3). MMP-2 (matrix-metalloproteinase 2) inhibitors, and MMP-9 (matrix-metalloproteinase 9) inhibitors.

Preferred VEGF inhibitors, include for example, Avastin (bevacizumab), an anti-VEGF monoclonal antibody of Genentech, Inc. of South San Francisco, Calif.

Additional VEGF signaling agents include CP-547,632 (Pfizer Inc., NY, USA), AG13736 (Pfizer Inc.), Vandetanib (Zactima), sorafenib (Bayer/Onyx), AEE788 (Novartis), AZD-2171, VEGF Trap (Regeneron/Aventis), vatalanib (also known as PTK-787. ZK-222584: Novartis & Schering AG as described in U.S. Pat. No. 6,258,812), Macugen (pegaptanib octasodium, NX-1838, EYE-001, Pfizer Inc./Gilead/Eyetech), IM862 (Cytran Inc. of Kirkland, Wash., USA); Neovastat (Aeterna); and Angiozyme (a synthetic ribozyme that cleaves mRNA producing VEGF1) and combinations thereof. VEGF inhibitors useful in the practice of the present invention are disclosed in U.S. Pat. No. 6,534,524 and U.S. Pat. No. 6,235,764, both of which are incorporated in their entirety for all purposed.

Particularly preferred VEGFR inhibitors include CP-547,632, AG-13736, AG-28262, Vatalanib, sorafenib, Macugen and combinations thereof.

Additional VEGFR inhibitors are described in, for example in U.S. Pat. No. 6,492,383, issued Dec. 10, 2002, U.S. Pat. No. 6,235,764 issued May 22, 2001, U.S. Pat. No. 6,177,401 issued Jan. 23, 2001, U.S. Pat. No. 6,395,734 issued May 28, 2002, U.S. Pat. No. 6,534,524 (discloses AG13736) issued Mar. 18, 2003, U.S. Pat. No. 5,834,504 issued Nov. 10, 1998, U.S. Pat. No. 6,316,429 issued Nov. 13, 2001, U.S. Pat. No. 5,883,113 issued Mar. 16, 1999, U.S. Pat. No. 5,886,020 issued Mar. 23, 1999, U.S. Pat. No. 5,792,783 issued Aug. 11, 1998, U.S. Pat. No. 6,653,308 issued Nov. 25, 2003, WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety.

PDGFr inhibitors include but not limited to those disclosed in International Patent Publication number WO 01/40217, published Jun. 7, 2001 and International Patent Publication number WO 2004/020431, published Mar. 11, 2004, the contents of which are incorporated in their entirety for all purposes.

Preferred PDGFr inhibitors include Pfizer's CP-673,451 and CP-868,596 and their pharmaceutically acceptable salts.

TIE-2 inhibitors include GlaxoSmithKline's benzimidazoles and pyridines including GW-697465A such as described in International Patent Publications WO 02/044156 published Jun. 6, 2002, WO 03/066601 published Aug. 14, 2003, WO 03/074515 published Sep. 12, 2003, WO 03/022852 published Mar. 20, 2003 and WO 01/37835 published May 31, 2001. Other TIE-2 inhibitors include Regeneron's biologicals such as those described in International Patent Publication WO 09/611,269 published Apr. 18, 1996, Amgen's AMG-386, and Abbott's pyrrolopyrimidines such as A-422885 and BSF-466895 described in International Patent Publications WO 09/955,335, WO 09/917,770, WO 00/075139, WO 00/027822, WO 00/017203 and WO 00/017202.

In another more specific embodiment of the present invention the anti-cancer agent used in conjunction with a compound of Formula I and pharmaceutical compositions described herein is where the anti-angiogenesis agent is a protein kinase C β such as enzastaurin, midostaurin, perifosine, staurosporine derivative (such as RO318425, RO317549, RO318830 or RO318220 (Roche)), teprenone (Selbex) and UCN-01 (Kyowa Hakko)

Examples of useful COX-II inhibitors which can be used in conjunction with a compound of Formula I and pharmaceutical compositions described herein include CELEBREX™ (celecoxib), parecoxib, deracoxib, ABT-963, COX-189 (Lumiracoxib), BMS 347070, RS 57067, NS-398, Bextra (valdecoxib), Vioxx (rofecoxib), SD-8381, 4-methyl-2-(3,4-dimethylphenyl)-1-(4-sulfamoyl-phenyl)-1H-pyrrole, 2-(4-ethoxyphenyl)-4-methyl-1-(4-sulfamoylphenyl)-1H-pyrrole, T-614, JTE-522, S-2474, SVT-2016, CT-3, SC-58125 and Arcoxia (etoricoxib). Additionally, COX-II inhibitors are disclosed in U.S. patent application Ser. Nos. 10/801,446 and 10/801,429, the contents of which are incorporated in their entirety for all purposes.

In one specific embodiment of particular interest the anti-tumor agent is celecoxib as disclosed in U.S. Pat. No. 5,466,823, the contents of which are incorporated by reference in its entirety for all purposes.

In another embodiment the anti-tumor agent is deracoxib as disclosed in U.S. Pat. No. 5,521,207, the contents of which are incorporated by reference in its entirety for all purposes.

Other useful anti-angiogenic inhibitors used in conjunction with a compound of Formula I and pharmaceutical compositions described herein include aspirin, and non-steroidal anti-inflammatory drugs (NSAIDs) which nonselectively inhibit the enzymes that make prostaglandins (cyclooxygenase I and II), resulting in lower levels of prostaglandins. Such agents include, but are not limited to, Aposyn (exisulind), Salsalate (Amigesic), Diflunisal (Dolobid), Ibuprofen (Motrin), Ketoprofen (Orudis), Nabumetone (Relafen), Piroxicam (Feldene), Naproxen (Aleve, Naprosyn), Diclofenac (Voltaren), Indomethacin (Indocin), Sulindac (Clinoril), Tolmetin (Tolectin), Etodolac (Lodine), Ketorolac (Toradol), Oxaprozin (Daypro) and combinations thereof.

Preferred nonselective cyclooxygenase inhibitors include ibuprofen (Motrin), nuprin, naproxen (Aleve), indomethacin (Indocin), nabumetone (Relafen) and combinations thereof.

MMP inhibitors include ABT-510 (Abbott), ABT 518 (Abbott), Apratastat (Amgen), AZD 8955 (AstraZeneca), Neovostat (AE-941), COL 3 (CollaGenex Pharmaceuticals), doxycycline hyclate. MPC 2130 (Myriad) and PCK 3145 (Procyon).

Other anti-angiogenic compounds include acitretin, angiostatin, aplidine, cilengtide, COL-3, combretastatin A-4, endostatin, fenretinide, halofuginone, Panzem (2-methoxyestradiol), rebimastat, removab, Revlimid, squalamine, thalidomide, ukrain, Vitaxin (alpha-v/beta-3 integrin), and zoledronic acid.

In another embodiment the anti-cancer agent is a so called signal transduction inhibitor. Such inhibitors include small molecules, antibodies, and antisense molecules. Signal transduction inhibitors include kinase inhibitors, such as tyrosine kinase inhibitors, serine/threonine kinase inhibitors. Such inhibitors may be antibodies or small molecule inhibitors. More specifically signal transduction inhibitors include farnesyl protein transferase inhibitors. EGF inhibitor, ErbB-1 (EGFR), ErbB-2, pan erb, IGF1R inhibitors, MEK, c-Kit inhibitors. FLT-3 inhibitors, K-Ras inhibitors, PI3 kinase inhibitors. JAK inhibitors, STAT inhibitors, Raf kinase inhibitors. Akt inhibitors, mTOR inhibitor. P70S6 kinase inhibitors and inhibitors of the WNT pathway and no called multi-targeted kinase inhibitors.

In another embodiment the anti-cancer signal transduction inhibitor is a farnesyl protein transferase inhibitor. Farnesyl protein transferase inhibitors include the compounds disclosed and claimed in U.S. Pat. No. 6,194,438, issued Feb. 27, 2002; U.S. Pat. No. 6,258,824, issued Jul. 10, 2001; U.S. Pat. No. 6,586,447, issued Jul. 1, 2003; U.S. Pat. No. 6,071,935, issued Jun. 6, 2000; and U.S. Pat. No. 6,150,377, issued Nov. 21, 2000. Other farnesyl protein transferase inhibitors include AZO-3409 (AstraZeneca), BMS-214662 (Bristol-Myers Squibb), Lonafarnib (Sarasar) and RPR-115135 (Sanofi-Aventis). Each of the foregoing patent applications and provisional patent applications is herein incorporated by reference in their entirety.

In another embodiment the anti-cancer signal transduction inhibitor is a GARF inhibitor. Preferred GARF inhibitors (glycinamide ribonucleotide formyltransferse inhibitors) include Pfizer's AG-2037 (pelitrexol) and its pharmaceutically acceptable salts. GARF inhibitors useful in the practice of the present invention are disclosed in U.S. Pat. No. 5,608,082 which is incorporated in its entirety for all purposed.

In another embodiment the anti-cancer signal transduction inhibitors used in conjunction with a compound of Formula I and pharmaceutical compositions described herein include ErbB-1 (EGFr) inhibitors such as Iressa (gefitinib, AstraZeneca), Tarceva (erlotinib or OSI-774, OSI Pharmaceuticals Inc.), Erbitux (cetuximab, Imclone Pharmaceuticals, Inc.), Matuzumab (Merck AG), Nimotuzumab, Panitumumab (Abgenix/Amgen), Vandetanib, hR3 (York Medical and Center for Molecular Immunology), TP-38 (IVAX), EGFR fusion protein. EGF-vaccine, anti-EGFr immunoliposomes (Hermes Biosciences Inc.) and combinations thereof.

Preferred EGFr inhibitors include Iressa (gefitinib), Erbitux, Tarceva and combinations thereof.

In another embodiment the anti-cancer signal transduction inhibitor is selected from pan erb receptor inhibitors or ErbB2 receptor inhibitors, such as CP-724,714, PF-299804, CI-1033 (canertinib, Pfizer, Inc.), Herceptin (trastuzumab, Genentech Inc.), Omnitarg (2C4, pertuzumab, Genentech Inc.), AEE-788 (Novartis), GW-572016 (Iapatinib, GlaxoSmithKline), Pelitinib (HKI-272), BMS-599626, PKI-166 (Novartis), dHER2 (HER2 Vaccine, Corixa and GlaxoSmithKline), Osidem (IDM-1), APC8024 (HER2 Vaccine, Dendreon), anti-HER2/neu bispecific antibody (Decof Cancer Center), B7.her2.IgG3 (Agensys), AS HER2 (Research Institute for Rad Biology & Medicine), trifunctional bispecific antibodies (University of Munich) and mAB AR-209 (Aronex Pharmaceuticals Inc) and mAB 2B-1 (Chiron) and combinations thereof.

Preferred erb selective anti-tumor agents include Herceptin, TAK-165, CP-724,714, ABX-EGF, HER3 and combinations thereof.

Preferred pan erb receptor inhibitors include GW572016, PF-299804, Pelitinib, and Omnitarg and combinations thereof.

Additional erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Pat. Nos. 6,465,449, and 6,284,764, and International Application No. WO 2001/98277 each of which are herein incorporated by reference in their entirety.

Various other compounds, such as styrene derivatives, have also been shown to possess tyrosine kinase inhibitory properties, and some of tyrosine kinase inhibitors have been identified as erbB2 receptor inhibitors. Other erbB2 Inhibitors are described in European patent publications EP 566,226 A1 (published Oct. 20, 1993), EP 602,851 A1 (published Jun. 22, 1994), EP 635,507 A1 (published Jan. 25, 1995), EP 635,498 A1 (published Jan. 25, 1995), and EP 520,722 A1 (published Dec. 30, 1992). These publications refer to certain bicyclic derivatives, in particular quinazoline derivatives possessing anti-cancer properties that result from their tyrosine kinase inhibitory properties. Also, World Patent Application WO 92/20642 (published Nov. 26, 1992), refers to certain bis-mono and bicyclic aryl and heteroaryl compounds as tyrosine kinase inhibitors that are useful in inhibiting abnormal cell proliferation. World Patent Applications WO96/16960 (published Jun. 6, 1996), WO 96/09294 (published Mar. 6, 1996), WO 97/30034 (published Aug. 21, 1997), WO 98/02434 (published Jan. 22, 1998), WO 98/02437 (published Jan. 22, 1998), and WO 98/02438 (published Jan. 22, 1998), also refer to substituted bicyclic heteroaromatic derivatives as tyrosine kinase inhibitors that are useful for the same purpose. Other patent applications that refer to anti-cancer compounds are World Patent Application WO00/44728 (published Aug. 3, 2000), EP 1029853A1 (published Aug. 23, 2000), and WO01/98277 (published Dec. 12, 2001) all of which are incorporated herein by reference in their entirety.

In another embodiment the anti-cancer signal transduction inhibitor is an IGF1R inhibitor. Specific IGF1R antibodies (such as CP-751871) that can be used in the present invention include those described in International Patent Application No. WO 2002/053596, which is herein incorporated by reference in its entirety.

In another embodiment the anti-cancer signal transduction inhibitor is a MEK inhibitor. MEK inhibitors include Pfizer's MEK1/2 inhibitor PD325901, Array Biopharma's MEK inhibitor ARRY-142886, and combinations thereof.

In another embodiment the anti-cancer signal transduction inhibitor is an mTOR inhibitor. mTOR inhibitors include everolimus (RAD001, Novartis), zotarolimus, temsirolimus (CCI-779, Wyeth), AP 23573 (Ariad), AP23675, Ap23841, TAFA 93, rapamycin (sirolimus) and combinations thereof.

In another embodiment the anti-cancer signal transduction inhibitor is an Aurora 2 inhibitor such as VX-680 and derivatives thereof (Vertex), R 763 and derivatives thereof (Rigel) and ZM 447439 and AZD 1152 (AstraZeneca), or a Checkpoint kinase ½ inhibitors such as XL844 (Exilixis).

In another embodiment the anti-cancer signal transduction inhibitor is an Akt inhibitor (Protein Kinase B) such as API-2, perifosine and RX-0201.

Preferred multitargeted kinase inhibitors include Sutent, (SU-11248), described in U.S. Pat. No. 6,573,293 (Pfizer, Inc, NY, USA) and imatinib mesylate (Gleevec).

Additionally, other targeted anti-cancer agents include the raf inhibitors sorafenib (BAY-43-9006, Bayer/Onyx), GV-1002, ISIS-2503, LE-AON and GI-4000.

The invention also relates to the use of the compounds of the present invention together with cell cycle inhibitors such as the CDK2 inhibitors ABT-751 (Abbott), AZD-5438 (AstraZeneca), Alvocidib (flavopiridol, Aventis), BMS-387,032 (SNS 032 Bristol Myers), EM-1421 (Erimos), indisulam (Esai), seliciclib (Cyclacel). BIO 112 (One Bio), UCN-01 (Kyowa Hakko), and AT7519 (Astex Therapeutics) and Pfizer's multitargeted CDK inhibitors PD0332991 and AG24322.

The invention also relates to the use of the compounds of the present invention together with telomerase inhibitors such as transgenic B lymphocyte immunotherapy (Cosmo Bioscience), GRN 163L (Geron), GV1001 (Pharmexa), RO 254020 (and derivatives thereof), and diazaphilonic acid.

Biological response modifiers (such as antibodies, immunotherapeutics and peptide mimics), are agents that modulate defense mechanisms of living organisms or biological responses, such as survival, growth, or differentiation of tissue cells to direct them to have anti-tumor activity.

Immunologicals including interferons and numerous other immune enhancing agents that may be used in combination therapy with compounds of formula I, optionally with one or more other agent include, but are not limited to interferon alpha, interferon alpha-2a, interferon, alpha-2b, interferon beta, interferon gamma-1a, interferon gamma-1b (Actimmune), or interferon gamma-n1. PEG Intron A, and combinations thereof. Other agents include interleukin 2 agonists (such as aldesleukin, BAY-50-4798, Ceplene (histamine dihydrochloride), EMD-273063, MVA-HPV-IL2, HVA-Muc-1-IL2, interleukin 2, teceleukin and Virulizin), Ampligen, Canvaxin, CeaVac (CEA), denileukin, filgrastim, Gastrimmune (G17DT), gemtuzumab ozogamicin, Glutoxim (BAM-002), GMK vaccine (Progenies), Hsp 90 inhibitors (such as HspE7 from Stressgen, AG-858, KOS-953, MVJ-1-1 and STA-4783), imiquimod, krestin (polysaccharide K), lentinan, Melacine (Corixa), MelVax (mitumomab), molgramostim, Oncophage (HSPPC-96), OncoVAX (including OncoVAX-CL and OncoVAX-Pr), oregovomab, sargramostim, sizofuran, tasonermin, TheraCys, thymalfasin, pemtumomab (Y-muHMFG1), picibanil, Provenge (Dendreon), ubenimex, WF-10 (Immunokine), Z-100 (Ancer-20 from Zeria), Lenalidomide (REVIMID, Celegene), thalomid (Thalidomide), and combinations thereof.

Anti-cancer agents capable of enhancing antitumor immune responses, such as CTLA4 (cytotoxic lymphocyte antigen 4) antibodies, and other agents capable of blocking CTLA4 may also be utilized, such as MDX-010 (Medarex) and CTLA4 compounds disclosed in U.S. Pat. No. 6,682,736. Additional, specific CTLA4 antibodies that can be used in the present invention include those described in U.S. Provisional Application 60/113,647 (filed Dec. 23, 1998), U.S. Pat. No. 6,682,736 both of which are herein incorporated by reference in their entirety.

In another embodiment of the present invention the anti-cancer agent used in conjunction with a compound of Formula I and pharmaceutical compositions described herein is a CD20 antagonist. Specific CD20 antibody antagonists that can be used in the present invention include rituximab (Rituxan), Zevalin (Ibritumonnab tiuxetan), Bexxar (131-I-tositumomab), Belimumab (LymphoStat-B), HuMax-CD20 (HuMax, Genmab), R 1594 (Roche Genentech), TRU-015 (Trubion Pharmaceuticals) and Ocrelizumab (PRO 70769).

In another embodiment of the present invention the anti-cancer agent used in conjunction with a compound of Formula I and pharmaceutical compositions described herein is a CD40 antagonist. Specific CD40 antibody antagonists that can be used in the present invention include CP-870893. CE-35593 and those described in International Patent Application No. WO 2003/040170 which is herein incorporated by reference in its entirety. Other CD40 antagonists include ISF-154 (Ad-CD154, Tragen), toralizumab, CHIR 12.12 (Chiron), SGN 40 (Seattle Genetics) and ABI-793 (Novartis).

In another embodiment of the present invention the anti-cancer agent used in conjunction with a compound of Formula I and pharmaceutical compositions described herein is a hepatocyte growth factor receptor antagonist (HGFr or c-MET).

Immunosuppressant agents useful in combination with the compounds of Formula I include epratuzumab, alemtuzumab, daclizumab, lenograstim and pentostatin (Nipent or Coforin).

The invention also relates to the use of the compounds of Formula I together with hormonal, anti-hormonal, anti-androgenal therapeutic agents such as anti-estrogens including, but not limited to fulvestrant, toremifene, raloxifene, lasofoxifene, letrozole (Femara, Novartis), anti-androgens such as bicalutamide, finasteride, flutamide, mifepristone, nilutamide, Casodex® (4′-cyano-3-(4-fluorophenylsulphonyl)-2-hydroxy-2-methyl-3′-(trifluoromethyl)-propionanilide, bicalutamide) and combinations thereof.

The invention also contemplates the use of the compounds of the present invention together with hormonal therapy, including but not limited to, exemestane (Aromasin, Pfizer. Inc.), Abarelix (Praecis), Trelstar, anastrozole (Arimidex, Astrazeneca), Atamestane (Biomed-777), Atrasentan (Xinlay), Bosentan, Casodex (AstraZeneca), doxercalcifero), fadrozole, formestane, gosrelin (Zoladex, AstraZeneca), Histrelin (histrelin acetate), letrozole, leuprorelin (Lupron or Leuplin, TAP/Abbott/Takeda), tamoxifen citrate (tamoxifen, Nolvadex, AstraZeneca), and combinations thereof.

The invention also contemplates the use of the compounds of the present invention together with gene silencing agents or gene activating agents such as histone deacetylase (HDAC) inhibitors such as suberolanilide hydroxamic acid (SAHA, Merck Inc./Aton Pharmaceuticals), depsipeptide (FR901228 or FK228), G2M-777, MS-275, pivaloyloxymethyl butyrate and PXD-101.

The invention also contemplates the use of the compounds of the present invention together with gene therapeutic agents such as Advexin (ING 201), TNFerade (GeneVec, a compound which express TNFalpha in response to radiotherapy), and RB94 (Baylor College of Medicine).

The invention also contemplates the use of the compounds of the present invention together with ribonucleases such as Onconase (ranpirnase).

The invention also contemplates the use of the compounds of the present invention together with antisense oligonucleotides such as bcl-2 antisense inhibitor Genasense (Oblimersen, Genta).

The invention also contemplates the use of the compounds of the present invention together with proteosomics such as PS-341 (MLN-341) and Velcade (bortezomib).

The invention also contemplates the use of the compounds of the present invention together with anti-vascular agents such as Combretastatin A4P (Oxigene).

The invention also contemplates the use of the compounds of the present invention together with traditional cytotoxic agents including DNA binding agents, mitotic inhibitors, alkylating agents, anti-metabolites, intercalating antibiotics, topoisomerase inhibitors and microtubulin inhibitors.

Topoisomerase I inhibitors useful in the combination embodiments of the present invention include 9-aminocamptothecin, belotecan, BN-80915 (Roche), camptothecin, diflomotecan, edotecarin, exatecan (Daiichi), gimatecan, 10-hydroxycamptothecin, irinotecan HCl (Camptosar), lurtotecan, Orathecin (rubitecan, Supergen), SN-38, topotecan, and combinations thereof.

Camptothecin derivatives are of particular interest in the combination embodiments of the invention and include camptothecin, 10-hydroxycamptothecin, 9-aminocamptothecin, irinotecan, SN-38, edotecarin, topotecan and combinations thereof.

A particularly preferred toposimerase I inhibitor is irinotecan HCl (Camptosar).

Topoisomerase II inhibitors useful in the combination embodiments of the present invention include aclarubicin, adriamycin, amonafide, amrubicin, annamycin, daunorubicin, doxorubicin, elsamitrucin, epirubicin, etoposide, idarubicin, galarubicin, hydroxycarbamide, nemorubicin, novantrone (mitoxantrone), pirarubicin, pixantrone, procarbazine, rebeccamycin, sobuzoxane, tafluposide, valrubicin, and Zinecard (dexrazoxane).

Particularly preferred toposimerase II inhibitors include epirubicin (Ellence), doxorubicin, daunorubicin, idarubicin and etoposide.

Alkylating agents that may be used in combination therapy with compounds of formula I, optionally with one or more other agents include, but are not limited to, nitrogen mustard N-oxide, cyclophosphamide, AMD-473, altretamine, AP-5280, apaziquone, brostallicin, bendamustine, busulfan, carboquone, carmustine, chlorambucil, dacarbazine, estramustine, fotemustine, glufosfamide, ifosfamide, KW-2170, lomustine, mafosfamide, mechlorethamine, melphalan, mitobronitol, mitolactol, mitomycin C, mitoxatrone, nimustine, ranimustine, temozolomide, thiotepa, and platinum-coordinated alkylating compounds such as cisplatin, Paraplatin (carboplatin), eptaplatin, lobaplatin, nedaplatin, Eloxatin (oxaliplatin, Sanofi), streptozocin, or satrplatin and combinations thereof.

Particularly preferred alkylating agents include Eloxatin (oxaliplatin).

Antimetabolites that may be used in combination therapy with compounds of formula I, optionally with one or more other agents include, but are not limited to dihydrofolate reductase inhibitors (such as methotrexate and NeuTrexin (trimetresate glucuronate)), purine antagonists (such as 6-mercaptopurine riboside, mercaptopurine, 6-thioguanine, cladribine, clofarabine (Clolar), fludarabine, nelarabine, and raltitrexed), pyrimidine antagonists (such as 5-fluorouracil (5-FU), Alimta (premetrexed disodium, LY231514, MTA), capecitabine (Xeloda), cytosine arabinoside, Gemzar (gemcitabine, Eli Lilly), Tegafur (UFT Orzel or Uforal and including TS-1 combination of tegafur, gimestat and otostat), doxifluridine, carmofur, cytarabine (including ocfosfate, phosphate stearate, sustained release and liposomal forms), enocitabine, 5-azacitidine (Vidaza), decitabine, and ethynylcytidine) and other antimetabolites such as eflornithine, hydroxyurea, leucovorin, nolatrexed (Thymitaq), triapine, trimetrexate, or for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid and combinations thereof.

In another embodiment the anti-cancer agent is a poly(ADP-ribose) polymerase-1 (PARP-1) inhibitor such as AG-014699, ABT-472, INO-1001, KU-0687 and GPI 18180.

Microtubulin inhibitors that may be used in combination therapy with compounds of formula I, optionally with one or more other agents include, but are not limited to ABI-007, Albendazole, Batabulin, CPH-82, EPO 906 (Novartis), discodermolide (XAA-296), Vinfunine and ZD-6126 (AstraZeneca).

Antibiotics that may be used in combination therapy with compounds of formula I, optionally with one or more other agent including, but are not limited to, intercalating antibiotics such as actinomycin D, bleomycin, mitomycin C, neocarzinostatin (Zinostatin), peplomycin, and combinations thereof.

Plant derived anti-tumor substances (also known as spindle inhibitors) that may be used in combination therapy with compounds of formula I, optionally with one or more other agent include, but are not limited to, mitotic inhibitors, for example vinblastine, vincristine, vindesine, vinorelbine (Navelbine), docetaxel (Taxotere). Ortataxel, paclitaxel (including Taxoprexin a DHA/paciltaxel conjugate) and combinations thereof.

Platinum-coordinated compounds include but are not limited to, cisplatin, carboplatin, nedaplatin, oxaliplatin (Eloxatin), Satraplatin (JM-216), and combinations thereof.

Particularly preferred cytotoxic agents include Camptosar, capecitabine (Xeloda), oxaliplatin (Eloxatin), Taxotere and combinations thereof.

Other antitumor agents include alitretinoin, I-asparaginase, AVE-8062 (Aventis), calcitriol (Vitamin D derivative), Canfosfamide (Telcyta, TLK-286), Cotara (131I chTNT 1/b), DMXAA (Antisoma), exisulind, ibandronic acid, Miltefosine, NBI-3001 (IL-4), pegaspargase, RSR13 (efaproxiral), Targretin (bexarotene), tazarotne (Vitamin A derivative), Tesmilifene (DPPE), Theratope, tretinoin, Trizaone (tirapazamine), Xcytrin (motexafin gadolinium) and Xyotax (polyglutamate paclitaxel), and combinations thereof.

In another embodiment of the present invention statins may be used in conjunction with a compound of Formula I and pharmaceutical compositions. Statins (HMG-CoA reducatase inhibitors) may be selected from the group consisting of Atorvastatin (Lipitor, Pfizer Inc.), Provastatin (Pravachol, Bristol-Myers Squibb), Lovastatin (Mevacor, Merck Inc.), Simvastatin (Zocor, Merck Inc.), Fluvastatin (Lescol, Novartis), Cerivastatin (Baycol, Bayer), Rosuvastatin (Crestor, AstraZeneca), Lovostatin and Niacin (Advicor, Kos Pharmaceuticals), derivatives and combinations thereof.

In a preferred embodiment the statin is selected from the group consisting of Atovorstatin and Lovastatin, derivatives and combinations thereof.

Other agents useful as anti-tumor agents include Caduet, Lipitor and torcetrapib.

Another embodiment of the present invention of particular interest relates to a method for the treatment of breast cancer in a human in need of such treatment, comprising administering to said human an amount of a compound of Formula I (including hydrates, solvates and polymorphs of said compound of Formula I or pharmaceutically acceptable salts thereof), in combination with one or more (preferably one to three) anti-cancer agents selected from the group consisting of trastuzumab (Herceptin), docetaxel (Taxotere), paclitaxel, capecitabine (Xeloda), gemcitabine (Gemzar), vinorelbine (Navelbine), exemestane (Aromasin), letrozole (Femara) and anastrozole (Arimidex).

Another embodiment of the present invention of particular interest relates to a method for the treatment of colorectal cancer in a human in need of such treatment, comprising administering to said human an amount of a compound of Formula I (including hydrates, solvates and polymorphs of said compound of Formula I or pharmaceutically acceptable salts thereof), in combination with one or more (preferably one to three) anti-cancer agents selected from the group consisting of capecitabine (Xeloda), irinotecan HCl (Camptosar), bevacizumab (Avastin), cetuximab (Erbitux), oxaliplatin (Eloxatin), premetrexed disodium (Alimta), vatalanib (PTK-787), Sutent, AG-13736, SU-14843, PD-325901, Tarceva, Iressa, Pelitinib, Lapatinib, Mapatumumab, Gleevec, BMS 184476, CCI-779, ISIS 2503, ONYX 015 and Flavopyridol, wherein the amounts of the compound of Formula I together with the amounts of the combination anticancer agents is effective in treating colorectal cancer.

Another embodiment of the present invention of particular interest relates to a method for the treatment of renal cell carcinoma in a human in need of such treatment, comprising administering to said human an amount of a compound of Formula I (including hydrates, solvates and polymorphs of said compound of Formula I or pharmaceutically acceptable salts thereof), in combination with one or more (preferably one to three) anti-cancer agents selected from the group consisting of capecitabine (Xeloda), interferon alpha, interleukin-2, bevacizumab (Avastin), gemcitabine (Gemzar), thalidomide, cetuximab (Erbitux), vatalanib (PTK-787), Sutent, AG-13736, SU-11248, Tarceva, Iressa, Lapatinib and Gleevec, wherein the amounts of the compound of Formula I together with the amounts of the combination anticancer agents is effective in treating renal cell carcinoma.

Another embodiment of the present invention of particular interest relates to a method for the treatment of melanoma in a human in need of such treatment, comprising administering to said human an amount of a compound of Formula I (including hydrates, solvates and polymorphs of said compound of Formula I or pharmaceutically acceptable salts thereof), in combination with one or more (preferably one to three) anti-cancer agents selected from the group consisting of interferon alpha, interleukin-2, temozolomide, docetaxel (Taxotere), paclitaxel, DTIC, PD-325901, Axitinib, bevacizumab (Avastin), thalidomide, sorafanib, vatalanib (PTK-787), Sutent, CpG-7909, AG-13736, Iressa, Lapatinib and Gleevec, wherein the amounts of the compound of Formula I together with the amounts of the combination anticancer agents is effective in treating melanoma.

Another embodiment of the present invention of particular interest relates to a method for the treatment of Lung cancer in a human in need of such treatment, comprising administering to said human an amount of a compound of Formula I (including hydrates, solvates and polymorphs of said compound of Formula I or pharmaceutically acceptable salts thereof), in combination with one or more (preferably one to three) anti-cancer agents selected from the group consisting of capecitabine (Xeloda), bevacizumab (Avastin), gemcitabine (Gemzar), docetaxel (Taxotere), paclitaxel, premetrexed disodium (Alimta), Tarceva, Iressa, and Paraplatin (carboplatin), wherein the amounts of the compound of Formula I together with the amounts of the combination anticancer agents is effective in treating Lung cancer.

In one preferred embodiment radiation can be used in conjunction with a compound of Formula I and pharmaceutical compositions described herein. Radiation may be administered in a variety of fashions. For example, radiation may be electromagnetic or particulate in nature. Electromagnetic radiation useful in the practice of this invention includes, but is not limited, to x-rays and gamma rays. In a preferable embodiment, supervoltage x-rays (x-rays>=4 MeV) may be used in the practice of this invention. Particulate radiation useful in the practice of this invention includes, but is not limited to, electron beams, protons beams, neutron beams, alpha particles, and negative pi mesons. The radiation may be delivered using conventional radiological treatment apparatus and methods, and by intraoperative and stereotactic methods. Additional discussion regarding radiation treatments suitable for use in the practice of this invention may be found throughout Steven A. Leibel et al., Textbook of Radiation Oncology (1998) (publ. W. B. Saunders Company), and particularly in Chapters 13 and 14. Radiation may also be delivered by other methods such as targeted delivery, for example by radioactive “seeds,” or by systemic delivery of targeted radioactive conjugates. J. Padawer et al., Combined Treatment with Radioestradiol lucanthone in Mouse C3HBA Mammary Adenocarcinoma and with Estradiol lucanthone in an Estrogen Bioassay, Int. J. Radiat. Oncol. Biol. Phys. 7:347-357 (1981). Other radiation delivery methods may be used in the practice of this invention.

The amount of radiation delivered to the desired treatment volume may be variable. In a preferable embodiment, radiation may be administered in amount effective to cause the arrest or regression of the cancer, in combination with a compound of Formula I and pharmaceutical compositions described herein.

In a more preferable embodiment, radiation is administered in at least about 1 Gray (Gy) fractions at least once every other day to a treatment volume, still more preferably radiation is administered in at least about 2 Gray (Gy) fractions at least once per day to a treatment volume, even more preferably radiation is administered in at least about 2 Gray (Gy) fractions at least once per day to a treatment volume for five consecutive days per week.

In a more preferable embodiment, radiation is administered in 3 Gy fractions every other day, three times per week to a treatment volume.

In yet another more preferable embodiment, a total of at least about 20 Gy, still more preferably at least about 30 Gy, most preferably at least about 60 Gy of radiation is administered to a host in need thereof.

In one more preferred embodiment of the present invention 14 GY radiation is administered.

In another more preferred embodiment of the present invention 10 GY radiation is administered.

In another more preferred embodiment of the present invention 7 GY radiation is administered.

In a most preferable embodiment, radiation is administered to the whole brain of a host, wherein the host is being treated for metastatic cancer.

Further, the invention provides a compound of the present invention alone or in combination with one or more supportive care products, e.g., a product selected from the group consisting of Filgrastim (Neupogen), ondansetron (Zofran), Fragmin, Procrit, Aloxi, Emend, or combinations thereof.

This invention also relates to a method for the treatment of a disease or condition selected from the group consisting of autoimmune diseases (such as rheumatoid arthritis, juvenile arthritis, type I diabetes, lupus, systemic lupus erythematosus, inflammatory bowel disease, optic neuritis, psoriasis, multiple sclerosis, polymyalgia rheumatica, uveitis, and vasculitis), acute and chronic inflammatory conditions (such as osteoarthritis, liver fibrosis, adult Respiratory Distress Syndrome, Respiratory Distress Syndrome of infancy, ischemia reperfusion injury, and glomerulonephritis), chronic pain conditions (such as neuropathic pain) allergic conditions (such as asthma and atopic dermatitis), chronic obstructive pulmonary disease, infection associated with inflammation (such as viral inflammation (including influenza and hepatitis) and Guillian-Barre syndrome syndrome), chronic bronchitis, xeno-transplantation, transplantation tissue rejection (chronic and acute), organ transplant rejection (chronic and acute), atherosclerosis, restenosis (including, but not limited to, restenosis following balloon and/or stent insertion), granulomatous diseases (including sarcoidosis, leprosy and tuberculosis), scleroderma, ulcerative colitis. Crohn's disease, and Alzheimer's disease, in a mammal, preferably a human, comprising administering to said mammal an amount of a compound of the Formula I, or a pharmaceutically acceptable salt thereof (including hydrates, solvates and polymorphs of said compound of Formula I or pharmaceutically acceptable salts thereof), that is effective in treating the disease or condition.

In one embodiment of this method, the disease or condition is selected from the group consisting of rheumatoid arthritis, juvenile arthritis, psoriasis, systemic lupus erythematosus, and osteoarthritis.

In another more specific embodiment of this method, the disease or condition is selected from the group consisting of rheumatoid arthritis and osteoarthritis.

In another embodiment of this method, the disease or condition is selected from the group consisting of chronic obstructive pulmonary disease, asthma acute respiratory distress syndrome, atherosclerosis, multiple sclerosis, and scleroderma.

Another embodiment of the invention is a method of preparing a compound of Formula I:

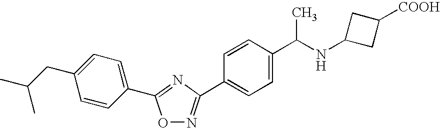

comprising hydrolyzing a compound of Formula II:

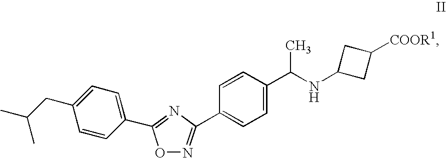

wherein R¹ is a C₁-C₆ alkyl.

As used herein, the term “alkyl,” may be linear or branched (such as methyl, ethyl, n-propyl, isopropyl, n-butyl, iso-butyl, secondary-butyl, tertiary-butyl), and they may also be cyclic (e.g., cyclopropyl or cyclobutyl) having the indicated number of carbon atoms. Preferred alkyls include (C₁-C₆)alkyl, most preferably methyl.

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; and (4) any tumors that proliferate by receptor tyrosine kinases.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

### DETAILED DESCRIPTION OF THE INVENTION

The compounds of the present invention are readily prepared according to synthetic methods familiar to those skilled in the art. Charts R and S illustrate general synthetic sequences for preparing compounds of the present invention.

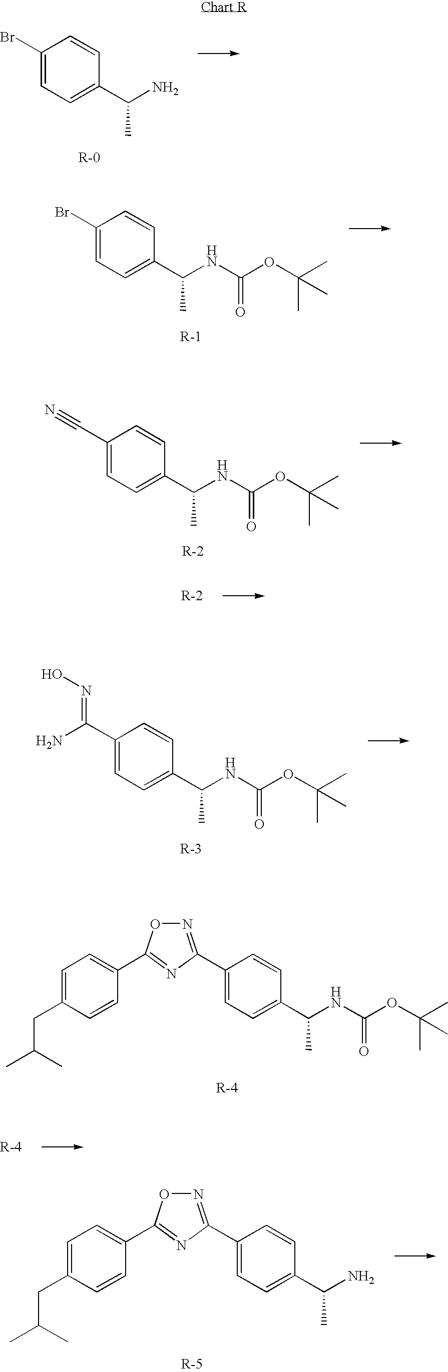

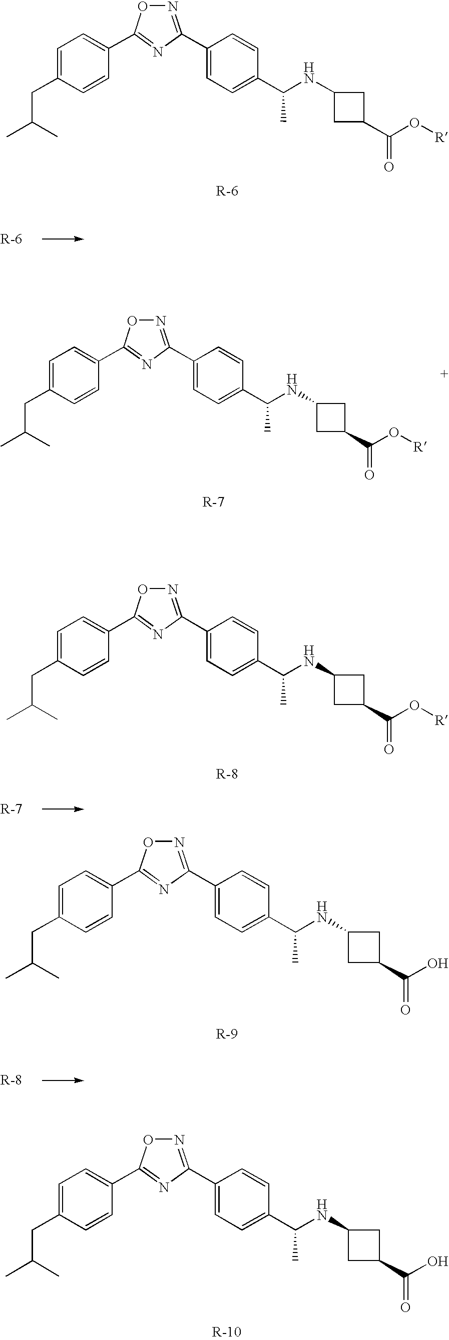

As shown in Chart R, (R)-(+)-1-(4-bromophenyl)ethylamine (CAS number 45791-36-4 commercially available from, for example, Sigma Aldrich Company, 3050 Spruce St. St. Louis, Mo. 63103 USA) (R-0) was protected using di-tert-butyl dicarbonate to give the tert-butyl carbamate of formula R-1. The reaction can be carried out in a suitable solvent, such as acetonitrile, tetrahydrofuran, chloroform, or dichloromethane, preferably dichloromethane. The reaction is typically performed at or below 22° C., preferably at 22° C. Additional conditions are known to those skilled in the art and can be found in Greene & Wuts, eds., Protecting Groups in Organic Synthesis, John Wiley & Sons, Inc.

The carbamate of formula R-1 was treated with zinc cyanide and palladium tetrakis(triphenylphosphine) to afford the cyano compound of formula R-2. The reaction can be performed in the presence of a suitable organometallic catalyst, and a suitable solvent, or mixture of solvents, at a temperature at or above 22° C. Suitable organometallic catalysts include, but are not limited to, tris(dibenzylidene acetone)dipalladium (Pd₂(dba)₃), palladium acetate (Pd(OAc)₂), and palladium tetrakis(triphenylphosphine); palladium tetrakis(triphenylphosphine) is preferred. The use of various suitable ligands for the catalyst may be needed to affect the aforementioned transformation efficiently. Suitable solvents include dimethylacetamide. N-methyl-pyrrolidinone, and dimethylformamide, preferably dimethylformamide. The nitrile can be prepared by methods well known to those skilled in the art (see Larock, Comprehensive Organic Transformations, A Guide to Functional Group Preparations, VCH publishers, Inc.).

The reaction of nitrile of formula R-2 with aqueous hydroxylamine gave the amine of formula R-3. The reaction can be performed in a suitable solvent or mixture of solvents. The reaction is carried out at or above 22° C. The reaction can be performed in a microwave at or above atmospheric pressure. Suitable solvents include methanol, isopropanol, and ethanol, preferably ethanol. Alternatively, the reaction can be preformed with hydroxylamine hydrochloride, and a suitable base in the presence of a suitable solvent or mixture of solvents. Suitable bases include sodium bicarbonate, triethylamine or diisopropylethylamine, preferably sodium bicarbonate. Suitable solvents include methanol, ethanol or dimethylformamide, preferably dimethylformamide. The reaction is carried out at or above 22° C.

The amine of formula R-3 was reacted with 4-isobutylbenzoyl chloride and cyclized to provide the oxadiazole compound of formula R-4. The acid chloride is prepared by methods known to those skilled in the art (see Larock, Comprehensive Organic Transformations, A Guide to Functional Group Preparations, VCH publishers, Inc.). The acylation reaction is typically carried out in the presence of a base and a suitable solvent or mixture of solvents. Suitable bases include pyridine, triethylamine, and diisopropylethylamine. Suitable solvents include pyridine, acetonitrile, tetrahydrofuran and dimethylformamide. Temperatures for the acylation reaction may be at or above 22° C., preferably 22° C. The cyclization/dehydration reaction is typically carried out using a suitable base and solvent (e.g., pyridine) at or above 22° C. to obtain the 1,2,4-oxadiazole. The reaction may be performed in a microwave at or above atmospheric pressure. Additional methods to prepare 1,2,4-oxadiazoles are potentially pertinent to the present invention and are known to those skilled in the art and have been reviewed in the literature (see Comprehensive Heterocyclic Chemistry, Volume 6, Potts, K. T., Editor, Pergamon Press, 1984).

Deprotection of the carbamate compound of formula R-4 with trifluoroacetic acid provides the amine of formula R-5. The reaction is typically carried out in the presence of a suitable organic co-solvent or mixture of solvents. Suitable solvents include 1,2-dichloroethane and dichloromethane, preferably dichloromethane. Temperatures for the reaction range from 0° C. to 22° C., preferably 22° C. Additional conditions for this transformation are known to those skilled in the art and can be found in Greene & Wuts, eds., Protecting Groups in Organic Synthesis, John Wiley & Sons, Inc.

Reductive amination of the amine of formula R-5 with various esters of 3-oxocyclobutanecarboxylate (wherein R′ includes, but is not limited to, methyl, ethyl, and t-butyl) afforded isomeric esters of formula R-6. Reductive aminations are typically carried out with a suitable reducing agent in the presence of a suitable solvent or mixture of solvents at a temperature from about −40° C. to about 50° C., preferably 22° C. Suitable reducing agents include sodium cyanoborohydride, sodium triacetoxyborohydride, and sodium borohydride. Sodium triacetoxyborohydride is preferred. Suitable solvents include methanol, ethanol, dichloroethane, tetrahydrofuran, methylene chloride and mixtures thereof, optionally in the presence of an acid or base, such as acetic acid or triethylamine, respectively. Esters of 3-oxocyclobutanecarboxylate can be prepared by methods well known to those skilled in the art (see J. Org. Chem. 1988 53, 3841-3843).

The isomeric esters of formula R-6 can be separated to obtain individual stereoisomers of formulas R-7 and R-8 by methods well known to those skilled in the art such as chromatography or recrystallization techniques. In addition, isomeric compounds of the invention and related precursors may be obtained in isomerically-enriched form using supercritical fluid chromatography (typically supercritical carbon dioxide) on an asymmetric resin with a mobile phase consisting of an alcohol, typically ethanol, 0 to 50% by volume, and supercritical carbon dioxide. Concentration of the product-containing fractions affords the isomerically-enriched material.

The hydrolysis of the ester of formulas R-7 and R-8 is typically carried out using acidic or basic conditions, optionally in the presence of a suitable organic co-solvent, e.g., methanol, ethanol, tetrahydrofuran, or dioxane. Suitable acids include hydrochloric acid or trifluoroacetic acid. Suitable bases include aqueous sodium, lithium or potassium hydroxide. Temperatures for the hydrolysis may range from about 0° C. to 120° C., more preferably about 22° C. Thus, the ester (e.g., methyl or ethyl) of formula R-7 can be hydrolyzed under basic conditions to provide the acid of formula R-9. In a similar fashion, lower alkyl esters of formula R-8 can be converted to the acid of formula R-10. Fort-butyl ester of formulas R-7 and R-8, removal under acidic conditions provides the acid of formulas R-9 and R-10 respectively.

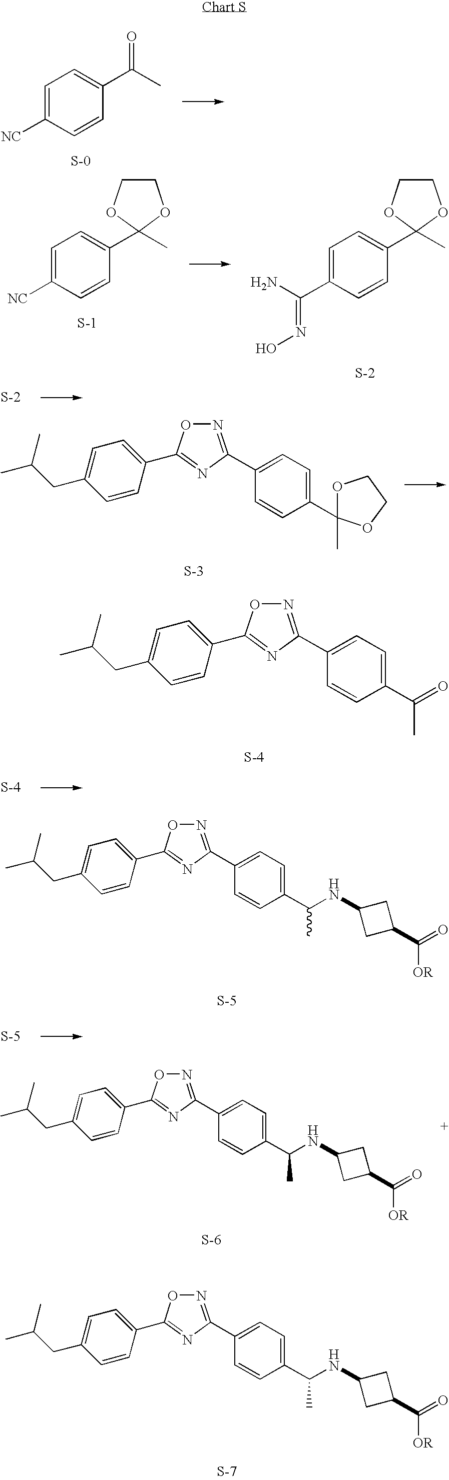

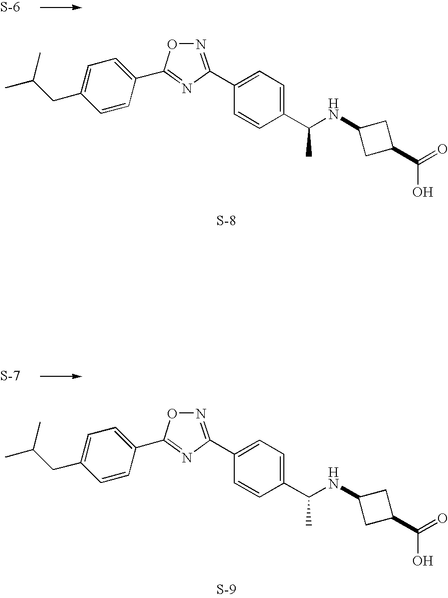

As shown in chart 8,4-cyanoacetophenone, commercially available from, for example, Sigma Aldrich Company, 3050 Spruce St., St. Louis, Mo. 63103 USA (S-0) was protected as its ethylene ketal. S-1, using ethylene glycol. The ketal formation is typically carried out using acidic conditions in the presence of a suitable organic co-solvent at or above 22° C. Suitable acid catalysts include para-toluenesulfonic acid, pyridinium para-toluenesulfonate, and boron trifluoride etherate, preferably boron trifluoride etherate. Suitable solvents include benzene and toluene, preferably toluene. Additional conditions for this transformation are known to those skilled in the art and can be found in Greene & Wuts, eds., Protecting Groups in Organic Synthesis, John Wiley & Sons, Inc.

The ketal compound of formula S-1 was reacted with hydroxylamine to give the hydroxyl amidine compound of formula S-2. The reaction may be performed with hydroxylamine hydrochloride, and a suitable base in the presence of a suitable solvent or mixture of solvents. Suitable bases include sodium bicarbonate, triethylamine, diisopropylethylamine, sodium or potassium hydroxide, preferably potassium hydroxide. Suitable solvents include ethanol, methanol or dimethylformamide, preferably methanol. The reaction is carried out at or above 22° C. Additional conditions for this transformation are described above.

The reaction of the hydroxylamine of formula S-2 with 4-isobutylbenzoic acid gave the oxadiazole compound of formula S-3. The oxadiazole of formula S-3 can be prepared in a two step procedure by a coupling reaction of the amine of formula S-2 with the requisite acid, followed by cyclization/dehydration at an elevated temperature. The coupling reaction is typically carried out using a suitable coupling agent in the presence of a suitable solvent or mixture of solvents. Suitable coupling agents include 1,1′-carbonyldiimidizole, N,N′-dicyclohexylcarbodiimide, 1-(3-dimethylaminopropyl)-3-ethylcarbodiimide and 1-(hydroxyl)benzotrazole, preferably 1,1′-carbonyldiimidizole. Suitable solvents include acetonitrile, tetrahydrofuran, dimethylformamide, and 1-methyl-2-pyrrolidinone. Temperatures for the coupling reaction may be at or above 22° C., preferably 22° C. The dehydration reaction is typically carried out at or above 22° C., preferably above 50° C. to 110° C. to obtain the 1,2,4-oxadiazole. Additional conditions for this transformation are described above.

Removal of the ethylene ketal protecting group from the compound of formula S-3 with aqueous hydrochloric acid provided the acetophenone of formula S-4. This transformation is typically carried out using aqueous acidic conditions optionally in the presence of a suitable organic co-solvent at or above 22° C. Suitable acid catalysts include para-toluenesulfonic acid, pyridinium para-toluenesulfonate, and hydrochloric acid, preferably hydrochloric acid. Suitable organic co-solvents include acetone, tetrahydrofuran and methanol. Additional conditions for this transformation are well known to those skilled in the art and can be found in Greene & Wuts, eds., Protecting Groups in Organic Synthesis, John Wiley & Sons, Inc.

Reductive amination of the acetophenone of formula S-4 with various 3-aminocyclobutanecarboxylate esters (wherein R includes, but is not limited to, methyl, ethyl, and t-butyl) gave rise to isomeric mixtures of the amine of formula S-5. This transformation can be carried out using a titanium reagent, preferably titanium ethoxide, and an organic solvent such as tetrahydrofuran, followed by addition of a reducing agent such as sodium borohydride. The reaction can be performed at or near 22° C. Additional reductive amination conditions are described above. The 3-aminocyclobutanecarboxylate esters are prepared using methods well known to those skilled in the art from the corresponding ketone described above and dibenzylamine. The benzyl groups are then removed under hydrogenation conditions using hydrogen gas and a catalyst such as palladium on carbon (Pd/C), palladium hydroxide (Pd(OH)₂) or platinum on carbon (Pt/C) in an appropriate solvent such as methanol, ethanol, tetrahydrofuran, or dioxane at or above atmospheric pressure and at a temperature from about 10° C. to about 60° C., preferably 22° C. The isomeric 3-aminocyclobutanecarboxylate esters can be separated by methods well known to those skilled in the art such as chromatography or recrystallization techniques.

Separation of the isomeric mixture of formula S-5 led to the isolation of enantiomerically-enriched ester of formulas S-6 and S-7. Separation of isomeric mixtures using supercritical fluid chromatography on an asymmetric resin column is described above.

The ester (e.g., methyl or ethyl) of formulas S-6 or S-7 can be hydrolyzed under basic conditions to provide the acid of formulas S-8 and S-9. For the t-butyl ester of formulas S-6 and S-7, removal under acidic conditions provides the acid of formulas S-8 and S-9 respectively. The conditions for the hydrolysis reaction are as described above.

Included within the scope of the present invention are all stereoisomers of formula I, and mixtures of one or more thereof. Also included are acid addition or base salts wherein the counterion is optically active, for example, d-lactate or l-lysine, or racemic, for example, dl-tartrate or dl-arginine.

Cis/trans isomers may be separated by conventional techniques well known to those skilled in the art, for example, chromatography and fractional crystallisation.

Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC).

Alternatively, the racemate (or a racemic precursor) may be reacted with a suitable optically active compound, for example, an alcohol, or, in the case where the compound of formula I contains an acidic or basic moiety, a base or acid such as 1-phenylethylamine or tartaric acid. The resulting diastereomeric mixture may be separated by chromatography and/or fractional crystallization and one or both of the diastereoisomers converted to the corresponding pure enantiomer(s) by means well known to a skilled person.

Chiral compounds of the invention (and chiral precursors thereof) may be obtained in enantiomerically-enriched form using chromatography, typically HPLC, on an asymmetric resin with a mobile phase consisting of a hydrocarbon, typically heptane or hexane, containing from 0 to 50% by volume of isopropanol, typically from 2% to 20%, and from 0 to 5% by volume of an alkylamine, typically 0.1% diethylamine. Concentration of the eluate affords the enriched mixture.

When any racemate crystallises, crystals of two different types are possible. The first type is the racemic compound (true racemate) referred to above wherein one homogeneous form of crystal is produced containing both enantiomers in equimolar amounts. The second type is the racemic mixture or conglomerate wherein two forms of crystal are produced in equimolar amounts each comprising a single enantiomer.

While both of the crystal forms present in a racemic mixture have identical physical properties, they may have different physical properties compared to the true racemate. Racemic mixtures may be separated by conventional techniques known to those skilled in the art—see, for example, Stereochemistry of Organic Compounds by E. L. Eliel and S. H. Wilen (Wiley, 1994).

The compounds of Formula I that are basic in nature are capable of forming a wide variety of different salts with various inorganic and organic acids. Although such salts must be pharmaceutically acceptable for administration to animals, it is often desirable in practice to initially isolate the compound of Formula I from the reaction mixture as a pharmaceutically unacceptable salt and then simply convert the later back to the free-base compound by treatment with an alkaline reagent and subsequently convert the latter free base to a pharmaceutically acceptable acid addition salt. The acid addition salt of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of Formula I that are acidic in nature are capable of forming base salts with various pharmacologically-acceptable cations. Examples of such salts include the alkali metal or alkaline-earth metal salts and particularly, the sodium and potassium salts. These salts are all prepared by conventional techniques. The chemical bases, which are used as reagents to prepare the pharmaceutically acceptable base salts of this invention, are those which form non-toxic, base salts with the acidic compounds of Formula I. Such non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together and then evaporating the resulting solution to dryness in the same manner as before. In either case, stoichiometric quantities of reagents are preferably employed in order to ensure completeness of reaction and maximum yields of the desired final product.

The compounds of the present invention are modulators of the S1P1 receptor, which is involved in angiogenesis/vasculogenesis, oncogenic and protooncogenic signal transduction and cell cycle regulations. As such, the compounds of the present invention are useful in the prevention and treatment of a variety of human hyperproliferative disorders, such as malignant and benign tumors of the liver, kidney, bladder, breast, gastric, ovarian, colorectal, prostate, pancreatic, lung, vulval, thyroid, hepatic carcinomas, sarcomas, glioblastomas, head and neck, and other hyperplastic conditions, such as benign hyperplasia of the prostate (e.g., BPH). It is, in addition, expected that a compound of the present invention may possess activity against a range of leukemias and lymphoid malignancies.

Further, it is expected that a compound of the present invention will possess activity in diseases or conditions such as autoimmune diseases and inflammation, for example as an analgesic in the treatment of pain and headaches, or as an antipyretic for the treatment of fever, and will be useful to treat arthritis, including but not limited to rheumatoid arthritis, spondyloarthropathies, gouty arthritis, osteoarthritis, systemic lupus erythematosus, juvenile arthritis, acute rheumatic arthritis, enteropathic arthritis, neuropathic arthritis, psoriatic arthritis, and pyogenic arthritis, type I diabetes, lupus, systemic lupus erythematosus, inflammatory bowel disease, optic neuritis, psoriasis, multiple sclerosis, polymyalgia rheumatica, uveitis, vasculitis, acute and chronic inflammatory conditions, osteoarthritis, adult Respiratory Distress Syndrome, Respiratory Distress Syndrome of infancy, ischemia reperfusion injury, glomerulonephritis, allergic conditions, asthma, atopic dermatitis, chronic obstructive pulmonary disease, infection associated with inflammation, viral inflammation, influenza, hepatitis. Guillian-Barre syndrome, chronic bronchitis, xeno-transplantation, transplantation tissue rejection (chronic and acute), organ transplant rejection (chronic and acute), atherosclerosis, restenosis, granulomatous diseases, sarcoidosis, leprosy, scleroderma, ulcerative colitis, Crohn's disease, and Alzheimer's disease.

Further, the present invention may have therapeutic utility in conditions or diseases associated with allergy/respiratory, cardiovascular, diabetes, endocrine care, frailty, obesity, neurodegeneration, dermatology, pain management, urology and sexual health, which may involve the S1P1 receptor that may be mediated by the compounds of this invention.

The activity of the compounds of the invention for the various disorders, diseases or conditions described above can be determined according to one or more of the following assays.

In addition, the compounds of the present invention may be evaluated for differential activity amongst the S1P receptor family members by the GTPγ35S method.

The in vitro activity of the compounds of Formula I and Ia in inhibiting the binding of S1P to the S1P1 receptor may be determined by the following procedure.

The in vitro activity of the compounds of Formula I in inhibiting the binding of S1P to the S1P₁ receptor may be determined by the following procedure.

Cell Transfection and Clonal Selection:

HEK293 or CHO cells expressing S1P₁₋₅, are prepared in ˜0.5×10⁵ cells/well. Cells are plated into each well of a 6-well plate in 2 ml of growth media (OptiMEM, Invitrogen). Two micrograms receptor plasmid DNA is mixed in 200 ul OptiMEM, and combined with 6 ul Lipofectamine (2000-9, Invitrogen). The mixture is added drop wise to 2 ml of growth media covering the cells in each well. The cells are allowed to transfect for 8-18 hours at room temperature. The OptiMEM transfection medium is replaced with 2 ml fresh serum-containing medium an incubated for 48 hours. The cells are diluted 1:10 in selection media (OptiMEM. Invitrogen) containing 0.8 mg/ml G418 in 10 cm dishes. Colonies are allowed to form (˜1-2 weeks), and 12 colonies from each dish are independently harvested with cloning disks and placed into 24-well plates.

Radioligand Binding Assay:

Cell membranes from CHO-S1P₁₋₅ or HEK-S1P₁₋₅ transfected cells are prepared by homogenizing the cells in an ice cold solution containing 25 mM Tris, 5 mM EDTA, 5 mM EGTA, and Complete Protease Inhibitor Cocktail, EDTA-Free (Roche #1 873 580). Cells are lysed by dounce homogenization and centrifugation at 20,000×g for 20 minutes at 4° C. Membrane pellets are resuspended in the same buffer and centrifuged again at 20,000×g for 20 minutes at 4° C. Final membrane pellet is resuspended in 20 mM HEPES, pH 7.5, 5 mM MgCl₂, 1 mM CaCl₂. Protein concentration is determined using the Micro BCA protein assay (Pierce #23235).

Serial dilutions of test compounds in DMSO are prepared in 96 well polypropylene plates. Using the FX robot 1:50 intermediate dilutions are made to assay buffer (20 mM HEPES, pH 7.5, 5 mM MgCl₂, 1 mM CaCl₂, 4 mg/ml fatty-acid free BSA). This intermediate is further diluted 1:10 to the final assay reaction. Final DMSO concentration in the reaction is 0.2%. Final reaction contains 50 pM ³³P-S1P (Perkin Elmer; Special Order), and 2.5 μg of cell membranes. The reaction is incubated at room temperature for 30 minutes and stopped by filtration through GF/B UniFilter Plates (Perkin Elmer #6005177) and washed four times with wash buffer containing 50 mM Tris pH 7.4, 0.025% Tween-20. The filter plates are dried for approximately 20 minutes in an oven at 50° C. Back seals are adhered to the filter plates and 40 μl of Microscint-20 scintillation fluid is added (Perkin Elmer #6013621). The filter plates are sealed, shaken for 30 minutes at room temperature, and counted on a Top Count (PerkinElmer).

GTPγ³⁵S Binding Assay

GTPγ³⁵S binding assays may be used to evaluate compound mediated S1P receptor agonism or antagonism. Cell membranes, are prepared as described above from CHO cells transfected with S1P receptors. Serial dilutions of test compounds in DMSO are prepared in 96 well polypropylene plates. Using the FX robot 1:50 intermediate dilutions are made to assay buffer (20 mM HEPES, pH 7.4, 100 mM NaCl, 10 mM MgCl₂, 0.2% fatty acid free BSA, and 10 μM GDP.). This intermediate is further diluted 1:10 to the final assay reaction. Final DMSO concentration in the reaction is 0.2%. 40 μl of test compound is incubated with 20 μl of [³⁵S] GTPgS (Perkin Elmer #NEG030H (1250 Ci/millimole)) and 140 μl of membrane homogenate (5 ug/well) in polypropylene 96-well plates (Corning #3365). Antagonism can be assessed by the addition of serial dilution of compounds added to membrane incubations containing EC80 concentrations of an agonist.

After incubation @ room temperature for 60 minutes reactions are harvested by vacuum filtration through Unifilter GF/B-96 filters (Perkin Elmer #6005177) using a FilterMate Plate Harvester (Perkin Elmer). Filters are washed 4 times with ice cold 50 mM Tris pH 7.4, 3 mM MgCl₂, 0.2 mM EGTA and dried at 50° C. for at least 30 minutes. 40 il of Microscint-20 (Perkin Elmer #6013621) is added per well, and plates are counted using a Top-Count Microplate Scintillation Counter (Perkin Elmer).

ERK Phosphorylation Assay

Phosphorylation of ERK may also be used to measure compound mediated S1P receptor agonism or antagonism.

Cell Culture

Cells are dispersed from frozen aliquots (1E+07 cells/vial stored in liquid nitrogen) into 25 ml of growth medium. (F12K Nutrient Mixture-Kaighn's Modified (catalog #21127-022). 1% penicillin-streptomycin (catalog #15140-122) purchased from Invitrogen Corp. (Madison, Wis.), and 10% fetal bovine serum (catalog #12103C) purchased from SAFC Biosciences (Lenexa, Kans.)) which contained the appropriate selection antibiotic: for S1P1-CHO clone C12 cells=10 μg/ml puromycin (catalog #P9620, Sigma-Aldrich), for S1P3-CHO cells=400 μg/ml geneticin (catalog #10131-027, Invitrogen Corp), and for S1P4-CHO cells=500 μg/ml geneticin. CHO-K1 cells (parental cell line) are dispersed in growth media without supplemental selection antibiotic. Cells are counted using a haemocytometer and volume adjusted to 100,000 cells/ml. The cells are plated to 384-well tissue culture plates (Becton Dickinson catalog #353962) at 40 μl/well (4000 cells/well) and the plates are incubated overnight in a humidified incubator under 5% CO₂ at 37° C. The cells are then serum starved by removing the growth media by aspiration and ishing once with 45 μl assay media (F12K Nutrient Mixture-Kaighn's Modified containing 0.1% fatty acid-free bovine serum albumin (catalog #009048-46-8, Sigma-Aldrich)). The plates are cultured overnight in 45 μl/well assay media in a humidified incubator under 5% CO₂ at 37° C.

Compound Treatment of Cells

All compounds are solubilized in 100% DMSO and 0.5 μl is spotted into 384 well polypropylene plates. Compounds are diluted with 50 μl assay media to give final DMSO concentrations of 1%. 5 μl of compound is added to 45 μl of cells using the Beckman Multimek workstation. The cell plate is then incubated at 37° C. for 5 minutes. The media is then removed by a rapid snap inversion of the plate and brief blotting of the top of the plate on a paper towel. 40 μl per well of 1× Lysis Buffer (TGR Surefire ERK1 384 Kit catalog #TGRES50K) is than added using a Titertek Multidrop dispenser. After agitation for 10 minutes at room temperature the plate is sealed and stored at −80° C. prior to lysate pERK analysis.

pERK ½ Alphascreen Assay

The cell lysates are thawed at 4° C. and the plates are spun for 5 minutes at 4° C. at 1000 rpm in a Beckman tabletop centrifuge. 20 μl of lysate is removed from each well and added to a Costar polypropylene 384 well plate using the Beckman Multimek workstation. 5 μl of Surefire pERK activation buffer is added to each well and mixed by gentle agitation on a plate shaker for 2 minutes. 5 μl of activated lysate from each well is then transferred to a 384-well Proxiplate (Perkin Elmer catalog #6008280). The Reaction Mix is prepared from the Alphascreen Protein A Detection Kit. The Anti-IgG (Protein A) and streptavidin beads are diluted 60× in Reaction Buffer under green light (they are extremely light sensitive). 6 μl of the working Reaction mix is added to each well under green light and the Proxiplate is sealed with an aluminum plate seal. The plate is shaken for 5 minutes after which it is stored at room temperature for at least 2 hours prior to reading on an AlphaQuest plate reader (Perkin Elmer).

TABLE I
Binding Affinities (Ki or IC50) at 33P-S1P labelled receptors

| | S1P₁ | S1P₂ | S1P₃ | S1P₄ | S1P₅ |
|---|---|---|---|---|---|
| | Kᵢ, | IC₅₀ | IC₅₀ | IC₅₀ | IC₅₀ |
| | nM | nM | nM | nM | nM |
| S1P | 0.16 | 30 | 7.1 | 15 | 0.25 |
| 3-{3-[5-(4-Isobutyl-phenyl)- | 0.32 | >1,000 | >1,000 | 11 | 2.3 |
| [1,2,4]oxadiazole-3-yl]-benzylamino}-cis- | | | | | |
| cyclobutanecarboxylic acid | | | | | |
| (Example 34B of U.S. serial no 11/746,314) | | | | | |
| cis-3-((R)-1-(4-(5-(4-isobutylphenyl)-1,2,4- | 0.29 | >1,000 | >1,000 | 35 | 2.9 |
| oxadiazol-3- | | | | | |
| yl)phenyl)ethylamino)cyclobutanecarboxylic | | | | | |
| acid | | | | | |
| cis-3-((S)-1-(4-(5-(4-isobutylphenyl)-1,2,4- | 2.7 | >1,000 | >1,000 | 331 | 11 |
| oxadiazol-3- | | | | | |
| yl)phenyl)ethylamino)cyclobutanecarboxylic | | | | | |
| acid | | | | | |
| trans-3-((R)1-(4-(5-(4-isobutylphenyl)-1,2,4- | 4.6 | >1,000 | >1,000 | 438 | 15 |
| oxadiazol-3- | | | | | |
| yl)phenyl)ethylamino)cyclobutanecarboxylic | | | | | |
| acid | | | | | |

Whole cell cAMP Flashplate Assay for Determining Functional Agonism:

The Perkin Elmer [FP]2 cAMPfire assay kit (Catalog #FPA20B040KT) is used to determine agonist potencies for S1P1 in whole cells.

1× cAMP antibody solution and 1× Alexa-Fluor is prepared as described in the cAMPfire assay protocol. The test compounds are dissolved in DMSO and then diluted to final concentrations about 9 nm to 0.0005 mM in the assay buffer, composed of 2 mg/ml FAF-BSA (final 1 mg/ml), 1 mM CaCl₂ (0.5 mM final), 5 mM MgCl₂ (2.5 mM final) in PBS. Ten microliters of the test compound dilutions are placed into 384-well assay plates. Ten microliters of buffer are placed in control wells. CHO-S1P1 transfected cells (90-100% confluent) are harvested using cell dissociation buffer (GIBCO, 13151-014). The cells are centrifuged, washed with PBS, counted, and resuspended in 1× cAMP antibody solution to achieve a final cell concentration of 3×10⁶ cells/well. Fifty-five mM of 11× forskolin solution (Sigma #F6886) in assay buffer is prepared. Ten microliters cells in 1× cAMP antibody are added to all applicable wells in 384-well assay plate. Two microliters of 55 μM forskolin (5 μM final in concentration) is added to all applicable wells in 384-well assay plate. Plates are incubated at room temperature for 30 minutes. Twenty microliters of 1× Alexa-Fluor are added to all wells followed by incubation for 60 minutes. Fluorescence polarization is read on Envison, (Perkin Elmer). A computerized algorithm gave the concentration of test compound that provided agonist activity greater than 40% at 9 μM.

The in vivo activity of the compounds of Formula I and Ia for inhibiting the S1P1 receptor may be determined by the following procedure.

Induction of Lymphopenia in Mice

S1P1 is expressed on the surface of T- and B-cells, and is necessary for S1P1/S1P mediated lymphocyte migration from secondary lymphoid tissue for release into peripheral circulation. Agonium of S1P1 results in S1P1 internalization, inhibiting lymphocyte egress into circulation, and is clinically presented as lymphopenia (Chiba, Pharmacology & Therapeutics 2005; 108,308-319, 2005). The following protocol may be used to assess the potential induction of lymphopenia for the test compounds when administered as a single oral dose to CD1 mice.

A suspension of 5% Gelucire may be used as the vehicle to prepare dosing formulations and to dose vehicle control animals. Test compound is weighed and transferred to a 15 mL Falcon tube or equivalent to make stock formulations. The appropriate amount of 5% Gelucire vehicle is then added to the tube. The resulting formulation is sonicated with a probe sonicator until no obvious particulate matter is apparent. About 500 mL Gelucire (Gattefosse, St-Priest, Cedex, France) is melted in a 1000 W microwave oven set for 3 minutes on high power. The appropriate amount of Gelucire is added to deionized water to form 5% (vol/vol) aqueous Gelucire.

Blood samples (˜0.6-0.8 mL) may be collected via intracardiac puncture at appropriate time points. The mice are anesthetized by carbon dioxide and euthanized via exanguination by intracardiac puncture. Blood samples are obtained and placed in tubes containing EDTA. Lymphocytes (L, %) count is measured with Abbott Cell-Dyn 3700 automated analyzer.

Induction of lymphopenia is calculated as a percent of the control count (% T/C), the ratio of the mean lymphocyte counts between treated mice and control mice. Based on the above, the ED₅₀ (the dose therapeutically effective in 50 percent of the population) can be determined by standard therapeutic procedures.

Samples of blood were taken to determine terminal half life (T½) and clearance of the compounds, using well accepted methods for such assays. The results of these assays are shown in Table II, below.

TABLE II
Clearance and Half-life of compounds

| | | T | Cl |
|---|---|---|---|
| Compound | Structure | T 1/2 h | (ml/min/kg) |
| 3-{3-[5-(4-Isobutyl- phenyl)-[1,2,4]oxadiazole-3- yl]-benzylamino}-cis- cyclobutanecarboxylic acid (Example 34B of U.S. Ser. No. 11/746,314) | 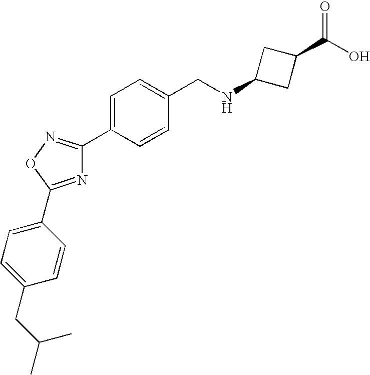 | 3.6 | 12.70 |
| cis-3-((S)-1-(4-(5-(4- isobutylphenyl)-1,2,4- oxadiazol-3- yl)phenyl)ethylamino)cyclo- butanecarboxylic acid; | 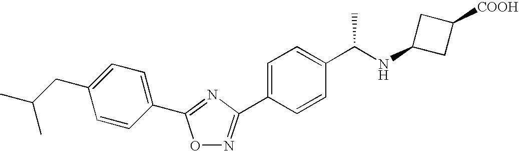 | 6.7 | 5.11 |
| cis-3-((R)-1-(4-(5-(4- isobutylphenyl)-1,2,4- oxadiazol-3- yl)phenyl)ethylamino)cyclo- butanecarboxylic acid | 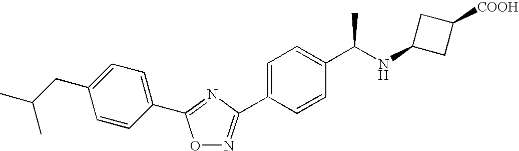 | 5.2 | 6.00 |

Inhibition of Growth Factor Induced Angiogenesis in Mice

The following protocol may be used to assess the potential inhibition of growth factor induced angiogenesis for the test compounds when administered as a single oral dose to CD1 mice.

A suspension of 5% Gelucire may be used as the vehicle to prepare dosing formulations and to dose vehicle control animals. Compound is weighed and transferred to a 15 mL Falcon tube or equivalent to make stock formulations. The appropriate amount of 5% Gelucire vehicle is then added to the tube. The resulting formulation is sonicated with a probe sonicator until no obvious particulate matter is apparent. About 500 mL Gelucire (Gattefosse, St-Priest, Cedex, France) is melted in a 1000 W microwave oven set for 3 minutes on high power. The appropriate amount of Gelucire is added to deionized water to form 5% (vol/vol) aqueous Gelucire.

Sterile porous Gelfrom absorbable gelatin sponges are cut to 3×3 mm pieces and filled with BD Matrigel Matrix (basement membrane preparation without phenol red from BD Bioscience Bedford Mass. #356237) with or without growth factor bFGF (recombinant bFGF 1 μg/plug; R&D Systems, Minneapolis, Minn.) and allowed to equilibrate for 2 hours. The sponges are implanted subcutaneous on the dorsal flank of mice. Animals are treated with the compounds of the present invention after sponge implantation and then once daily for a further 5 days. On the seventh day after implantation, animals are sacrificed, and the vascularized sponges are removed.

The sponge samples are harvested and ground with 200 μL sterile water and centrifuged for 10 minutes at 14,000 RPM. One hundred microliters of sample is removed and placed into a 96-well flat-bottom Falcon plate from BD Bioscience Bedford, Mass. One hundred microliters of TMB substrate (SureBlue TMB Microwell peroxidase substrate, KPL Gaithersburg, Md.) is added to all wells and allowed to incubate for 5 minutes. Fifty microliters of Stop solution (1NH₂SO₄) is added to all wells and absorbance is read at 450 nm with 750 nm correction on a VersaMax visible plate reader (Molecular Devices, Sunnyvale, Calif.).

Inhibition of angiogenesis is calculated as a percent of the control absorbance (% T/C), ratio of the mean absorbance between treated mice and control mice. Based on the above, the ED₅₀ can be determined by standard therapeutic procedures.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active compound administered will be dependent on the subject being treated, the severity of the disease, disorder or condition, the rate of administration and the judgment of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.005 to about 1 g/day, preferably about 0.05 to about 1 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The active compound may be applied as a sole therapy or may involve one or more other anti-tumor substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-(N-(3,4-dihydro-2-methyl-4-oxoquinazolin-6-ylmethyl)-N-methylamino]-2-thenoyl)-L-glutamic acid; growth factor inhibitor; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex (4′-cyano-3-(4-fluorophenylsulphonyl)-2-hydroxy-2-methyl-3′-trifluoromethyl)propionanilide). Such conjoint treatment may be achieved by way of simultaneous, sequential or separate dosing of the individual components of the treatment.

The pharmaceutical composition may, for example, be in a form suitable for oral administration as a tablet, capsule, pill, powder, sustained release formulations, solution, and suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like.

The compounds of the invention may also be administered topically to the skin or mucosa, that is, dermally or transdermally. Typical formulations for this purpose include gels, hydrogels, lotions, solutions, creams, ointments, dusting powders, dressings, foams, films, skin patches, wafers, implants, sponges, fibres, bandages and microemulsions. Liposomes may also be used. Typical carriers include alcohol, water, mineral oil, liquid petrolatum, white petrolatum, glycerin, polyethylene glycol and propylene glycol. Penetration enhancers may be incorporated [see, for example, J Pharm Sci, 88 (10), 955-958 by Finnin and Morgan (October 1999).]

Other means of topical administration include delivery by electroporation, iontophoresis, phonophoresis, sonophoresis and microneedle or needle-free (e.g. Powderject™, Bioject™, etc.) injection.

The compounds of the invention can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurised container, pump, spray, atomiser (preferably an atomiser using electrohydrodynamics to produce a fine mist), or nebuliser, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin.

The pressurised container, pump, spray, atomizer, or nebuliser contains a solution or suspension of the compounds of the invention comprising, for example, ethanol, aqueous ethanol, or a suitable alternative agent for dispersing, solubilising, or extending release of the active, a propellant(s) as solvent and an optional surfactant, such as sorbitan trioleate, oleic acid, or an oligolactic acid.

Prior to use in a dry powder or suspension formulation, the drug product may be micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenisation, or spray drying.

The compounds of the invention may be administered orally. Oral administration may involve swallowing, so that the compound enters the gastrointestinal tract, and/or buccal, lingual, or sublingual administration by which the compound enters the blood stream directly from the mouth.

Formulations suitable for oral administration include solid, semi-solid and liquid systems such as tablets; soft or hard capsules containing multi- or nano-particulates, liquids, or powders; lozenges (including liquid-filled); chews; gels; fast dispersing dosage forms; films; ovules; sprays; and buccal/mucoadhesive patches.

Liquid formulations include suspensions, solutions, syrups and elixirs. Such formulations may be employed as fillers in soft or hard capsules (made, for example, from gelatin or hydroxypropylmethylcellulose) and typically comprise a carrier, for example, water, ethanol, polyethylene glycol, propylene glycol, methylcellulose, or a suitable oil, and one or more emulsifying agents and/or suspending agents. Liquid formulations may also be prepared by the reconstitution of a solid, for example, from a sachet.

The compounds of the invention may also be used in fast-dissolving, fast-disintegrating dosage forms such as those described in Expert Opinion in Therapeutic Patents, 11 (6), 981-986, by Liang and Chen (2001).

For tablet dosage forms, depending on dose, the drug may make up from 1 weight % to 80 weight % of the dosage form, more typically from 5 weight % to 60 weight % of the dosage form. In addition to the drug, tablets generally contain a disintegrant. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, lower alkyl-substituted hydroxypropyl cellulose, starch, pregelatinised starch and sodium alginate. Generally, the disintegrant will comprise from 1 weight % to 25 weight %, preferably from 5 weight % to 20 weight % of the dosage form.

Binders are generally used to impart cohesive qualities to a tablet formulation. Suitable binders include microcrystalline cellulose, gelatin, sugars, polyethylene glycol, natural and synthetic gums, polyvinylpyrrolidone, pregelatinised starch, hydroxypropyl cellulose and hydroxypropyl methylcellulose. Tablets may also contain diluents, such as lactose (monohydrate, spray-dried monohydrate, anhydrous and the like), mannitol, xylitol, dextrose, sucrose, sorbitol, microcrystalline cellulose, starch and dibasic calcium phosphate dihydrate.

Tablets may also optionally comprise surface active agents, such as sodium lauryl sulfate and polysorbate 80, and glidants such as silicon dioxide and talc. When present, surface active agents may comprise from 0.2 weight % to 5 weight % of the tablet, and glidants may comprise from 0.2 weight % to 1 weight % of the tablet.

Tablets also generally contain lubricants such as magnesium stearate, calcium stearate, zinc stearate, sodium stearyl fumarate, and mixtures of magnesium stearate with sodium lauryl sulphate. Lubricants generally comprise from 0.25 weight % to 10 weight %, preferably from 0.5 weight % to 3 weight % of the tablet.

Other possible ingredients include anti-oxidants, colourants, flavouring agents, preservatives and taste-masking agents.

Exemplary tablets contain up to about 80% drug, from about 10 weight % to about 90 weight % binder, from about 0 weight % to about 85 weight % diluent, from about 2 weight % to about 10 weight % disintegrant, and from about 0.25 weight % to about 10 weight % lubricant.

Tablet blends may be compressed directly or by roller to form tablets. Tablet blends or portions of blends may alternatively be wet-, dry-, or melt-granulated, melt congealed, or extruded before tabletting. The final formulation may comprise one or more layers and may be coated or uncoated; it may even be encapsulated.

The formulation of tablets is discussed in Pharmaceutical Dosage Forms: Tablets, Vol. 1, by H. Lieberman and L. Lachman (Marcel Dekker, New York, 1980).

Consumable oral films for human or veterinary use are typically pliable water-soluble or water-swellable thin film dosage forms which may be rapidly dissolving or mucoadhesive and typically comprise a compound of formula I, a film-forming polymer, a binder, a solvent, a humectant, a plasticiser, a stabiliser or emulsifier, a viscosity-modifying agent and a solvent. Some components of the formulation may perform more than one function.

The compound of formula I may be water-soluble or insoluble. A water-soluble compound typically comprises from 1 weight % to 80 weight %, more typically from 20 weight % to 50 weight %, of the solutes. Less soluble compounds may comprise a greater proportion of the composition, typically up to 88 weight % of the solutes. Alternatively, the compound of formula I may be in the form of multiparticulate beads.

The film-forming polymer may be selected from natural polysaccharides, proteins, or synthetic hydrocolloids and is typically present in the range 0.01 to 99 weight %, more typically in the range 30 to 80 weight %.

Other possible ingredients include anti-oxidants, colorants, flavourings and flavour enhancers, preservatives, salivary stimulating agents, cooling agents, co-solvents (including oils), emollients, bulking agents, anti-foaming agents, surfactants and taste-masking agents.

Films in accordance with the invention are typically prepared by evaporative drying of thin aqueous films coated onto a peelable backing support or paper. This may be done in a drying oven or tunnel, typically a combined coater dryer, or by freeze-drying or vacuuming.

Solid formulations for oral administration may be formulated to be immediate and/or modified release. Modified release formulations include delayed-, sustained-, pulsed-, controlled-, targeted and programmed release.

Suitable modified release formulations for the purposes of the invention are described in U.S. Pat. No. 6,106,864. Details of other suitable release technologies such as high energy dispersions and osmotic and coated particles are to be found in Pharmaceutical Technology On-line, 25(2), 1-14, by Verma et al (2001). The use of chewing gum to achieve controlled release is described in WO 00/35298.

Methods of preparing various pharmaceutical compositions with a specific amounts of an active compound are known, or will be apparent to those skilled n this art. For example, see Remington's Pharmaceutical Sciences, Mack Publishing Company, Easter, Pa., 15^{th} Edition (1975).

The examples and preparations provided below further illustrate and exemplify the compounds of the present invention and methods of preparing such compounds. It is to be understood that the scope of the present invention is not limited in any way by the scope of the following examples and preparations.

Inasmuch as it may desirable to administer a combination of active compounds, for example, for the purpose of treating a particular disease or condition, it is within the scope of the present invention that two or more pharmaceutical compositions, at least one of which contains a compound in accordance with the invention, may conveniently be combined in the form of a kit suitable for coadministration of the compositions.

Thus the kit of the invention comprises two or more separate pharmaceutical compositions, at least one of which contains a compound of formula I in accordance with the invention, and means for separately retaining said compositions, such as a container, divided bottle, or divided foil packet. An example of such a kit is the familiar blister pack used for the packaging of tablets, capsules and the like.

The kit of the invention is particularly suitable for administering different dosage forms, for example, oral and parenteral, for administering the separate compositions at different dosage intervals, or for titrating the separate compositions against one another. To assist compliance, the kit typically comprises directions for administration and may be provided with a so-called memory aid.

The examples and preparations provided below further illustrate and exemplify the compounds of the present invention and methods of preparing such compounds. Alternative routes will be easily discernible to practitioners in the field. It is to be understood that the scope of the present invention is not limited in any way by the scope of the following examples and preparations.

### General

The following examples are put forth so as to provide those of ordinary skill in the art with a disclosure and description of how the compounds, compositions, and methods claimed herein are made and evaluated, and are intended to be purely exemplary of the invention and are not intended to limit the scope of what the inventors regard as their invention. Unless indicated otherwise, percent is percent by weight given the component and the total weight of the composition, temperature is in ° C. or is at ambient or room temperature (20-25° C.) and pressure is at or near atmospheric. Commercial reagents were utilized without further purification. Conventional flash chromatography was carried out on silica gel (230-400 mesh) and executed under nitrogen or air pressure conditions. Flash chromatography was also carried out using a Combi Flash Chromatography apparatus (Teledyne Isco Tech. Corp.) on silica gel (75-150 uM) in pre-packed cartridges. Particle Beam Mass Spectra were recorded on either a Hewlett Packard 59890, utilizing chemical ionization (ammonium), or a Fisons (or MicroMass) Atmospheric Pressure Chemical Ionization (APCI) platform which uses a 50/50 mixture of acetonitrile/water. NMR spectra were obtained using a Unity Inova Varian, 400 or 500 MHz, unless otherwise indicated. Chemical shifts are reported in parts per million (ppm) and coupling constants (J) in hertz (Hz). All non-aqueous reactions were run under a nitrogen atmosphere for convenience and to maximize yields. Concentration in vacuo means that a rotary evaporator under reduced pressure was used.

Abbreviations: ethyl acetate (EtOAc), tetrahydrofuran (THF), dimethylformamide (DMF), tetrabutylammonium fluoride (TBAF), 1,1,1-tris(acetyloxy)-1,1-dihydro-1,2-benziodoxol-3-(1H)-one [Dess Martin reagent (periodinane)], methanol (MeOH), ethanol (EtOH), ethyl (Et), acetyl (Ac), methyl (Me), and butyl (Bu).

### Preparation 1

### cis-3-Amino-cyclobutanecarboxylic acid ethyl ester, hydrochloride

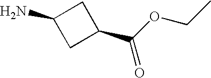

### Step 1A. 3-Oxo-cyclobutanecarboxylic acid ethyl ester

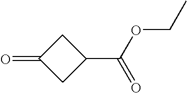

A solution of 3-oxo-cyclobutanecarboxylic acid (6.0 g, 52.4 mmol; J. Org. Chem. 1988 53, 3841-3843), triethylorthoacetate (28.8 mL, 157 mmol) and toluene (120 mL) was heated at 110° C. for 5 hours. The reaction mixture was cooled to room temperature and quenched with 1.0 N HCl (120 mL). The organic phase was separated, washed with a saturated NaHCO₃ and brine, dried (Na₂SO₄), filtered and concentrated in vacuo to provide the title compound (6.5 g, 80% yield) as an oil.

¹H NMR (400 MHz, DMSO-d₄) [symbol] 1.23 (t, 3H), 3.30 (m, 5H), 4.14 (q, 2H).

### Step 1B. 3-Dibenzylamino-cyclobutanecarboxylic acid ethyl ester

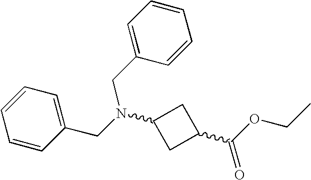

Dibenzyl amine (0.150 g, 0.77 mmol) and sodium triacetoxyborohydride (0.300 g, 1.4 mmol) were added to a solution of 3-oxo-cyclobutanecarboxylic acid ethyl ester (0.100 g, 0.700 mmol) and acetic acid/THF (10%, 4.4 mL), stirred at room temperature for 72 hours and concentrated in vacuo. The resulting residue was dissolved in dichloromethane, washed with water, saturated NaHCO₃ and brine, dried (Na₂SO₄) and concentrated in vacuo to give crude product. Purification by flash chromatography (silica, 1:9-3:7 EtOAc:hexanes) provided the title compound (0.180 g, 73% yield, 10:1 cis:trans ratio) as a solid.

¹H NMR (400 MHz, CD₃OD) [symbol] 1.22 (t, 3H), 2.08 (m, 2H), 2.20 (m, 2H), 2.70 (m, 1H), 3.11 (m, 1H), 3.50 (s, 4H), 4.09 (q, 2H), 7.30 (m, 10H); ESI-MS: 323 (MH⁺).

### Step 1C. cis-3-Amino-cyclobutanecarboxylic acid ethyl ester, hydrochloride

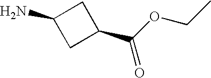

Pd/C (10% by wt, 0.50 g, 0.30 mmol) was added to a solution of 3-dibenzylamino-cyclobutanecarboxylic acid ethyl ester (1.0 g, 3.09 mmol), ethanol (48.0 mL), water (3.0 mL) and acetic acid (0.20 mL, 3.09 mmol) in a Parr shaker bottle. The reaction mixture was pressurized to 45 psi with H₂ and agitated at room temperature for 12 hours. The reaction mixture was filtered and the filtrate was concentrated in vacuo. The resulting residue was taken up in ethanol (2.0 mL) and HCl (2 M in diethyl ether, 0.77 mL) was added. The slurry was filtered to provide a crude solid (0.30 g). The solid was recrystallized from isopropyl alcohol (4.0 mL) to provide the title compound (0.100 g, 45% yield).

¹H NMR (400 MHz, CD₃OD) [symbol] 1.23 (t, 3H), 2.31 (m, 2H), 2.57 (m, 2H), 3.03 (m, 1H), 4.12 (q, 2H); ESI-MS: 144 (MH⁺).

### Preparation 2

### trans-3-Amino-cylcobutanecarboxylic acid ethyl ester, hydrochloride

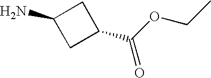

3-Dibenzylamino-cyclobutanecarboxylic acid ethyl ester (mixture of cis/trans) was loaded on a 2×25 cm Chiralpak AD-H preparatory HPLC column (UV detection @ 210 nM) using a 85:15 (vol:vol) mixture of heptane:ethanol as the mobile phase at a rate of 10 mL/min. The eluent containing the faster-eluting (Rf: 19.74 min) isomer was concentrated in vacuo. The residue was treated with Pd/C by procedures analogous to those described in Preparation 1C for the preparation of cis-3-amino-cylcobutanecarboxylic acid ethyl ester, hydrochloride to provide the title compound.

¹H NMR (400 MHz, CD₃OD) [symbol] 4.13 (q, J=0.83 Hz, 2H), 3.74-3.68 (m, 1H), 3.04-3.00 (m, 1H), 2.62-2.55 (m, 2H), 2.36-2.29 (m, 2H), 1.24 (t, J=0.83 Hz, 3H); ESI-MS: 144 (MH⁺).

### Preparation 3

### tert-Butyl [(1R)-1-(4-bromophenyl)ethyl]carbamate

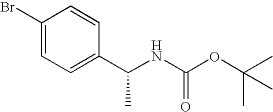

To a flask was charged (R)-(+)-1-(4-bromophenyl)ethylamine (11.5 gm), dichloromethane (200 mL), triethylamine (8.8 mL), and di-tert-butyl dicarbonate (13.6 gm). The mixture was stirred under a nitrogen atmosphere for 2 hours. The reaction was diluted with dichloromethane (450 mL) and washed successively with 1N aqueous HCl solution (300 mL), saturated aqueous sodium bicarbonate solution (250 mL), and brine (250 mL). The organic layer was dried over magnesium sulfate, filtered and concentrated under reduced pressure. The residue was slurried with heptane (200 mL) for 1 hr, then the solid collected by filtration and dried in a vacuum oven (40° C.) to provide the title compound as a white solid (16.3 gm): ¹H NMR (400 MHz, DMSO-d₆) δ ppm 7.46-7.53 (2H, m), 7.41 (1H, d), 7.20-7.28 (2H, m), 4.51-4.63 (1H, m), 1.35 (9H, s), 1.27 (3H, d).

### Preparation 4

### tert-Butyl [(1R)-1-(4-cyanophenyl)ethyl]carbamate

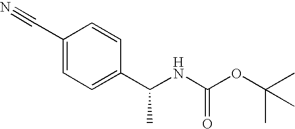

To a vial was charged tert-butyl [(1R)-1-(4-bromophenyl)ethyl]carbamate (4.3 gm), zinc cyanide (1.18 gm), and dimethylfomamide (13 mL). The mixture was purged with a stream of nitrogen than stirred under a nitrogen atmosphere for 1 hour. The reaction was then treated with palladium tetrakis(triphenylphosphine) (0.5 gm), sealed, and heated to 75° C. for 3.5 hours. The reaction was then heated at 70° C. for 17 hours. The mixture was cooled to room temperature and diluted with toluene (50 mL). Thiocyanuric acid (0.26 gm) was added followed by 3% aqueous sodium hydroxide (70 mL). The organic phase was washed again with a solution of thiocyanuric acid (0.26 gm) in 3% aqueous sodium hydroxide (70 mL). The organic phase was dried over sodium sulfate, filtered through a pad of Celite, concentrated under reduced pressure, and finally in vacuo to give a dark oil (3.9 gm). This material was diluted with dichloromethane and purified on a Biotage 65i column by elution with a gradient of 0-80% ethyl acetate/heptanes to give the title compound as a colorless oil (3.0 gm): ¹H NMR (400 MHz, CDCl₃) δ ppm 7.52-7.69 (2H, m), 7.41 (2H, m), 4.83 (2H, m), 1.21-1.52 (12H, m).

### Preparation 5

### tert-Butyl [(1R)-1-{4-[(hydroxyamino)(imino)methyl]phenyl}ethyl]carbamate

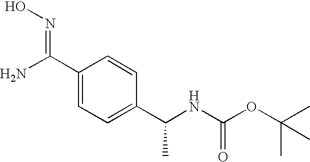

tert-Butyl [(1R)-1-(4-cyanophenyl)ethyl]carbamate (2.1 gm) was dissolved in ethanol (13 mL) and transferred to a microwave vial (20 mL). Aqueous hydroxylamine (50%, 0.63 mL) was added and the mixture was irradiated at 100° C. for 20 min. Additional hydroxylamine (0.32 mL) was added and the vial irradiated at 100° C. for 10 min. The reaction mixture was cooled to room temperature, treated with water (10 mL), stirred for 1 h, and then filtered. The collected white solid was dried in vacuo at 40° C. to give the title compound (2.14 g): ¹H NMR (400 MHz, DMSO-d₆) δ ppm 9.54 (1H, s), 7.59 (2H, d), 7.38 (1H, d), 7.27 (2H, d), 4.51-4.69 (1H, m), 1.36 (9H, s), 1.29 (3H, d).

### Preparation 6

### (1R)-1-{-4-[5-(4-Isobutylphenyl)-1,2,4-oxadiazol-3-yl]phenyl}ethanamine

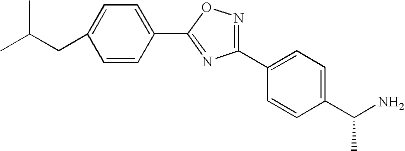

### Step 6A: tert-Butyl [(1R)-1-{4-[5-(4-isobutylphenyl)-1,2,4-oxadiazol-3-yl]phenyl}ethyl]carbamate

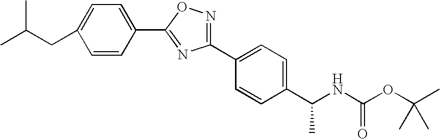

To a pyridine solution (17 ml) of tert-butyl [(1R)-1-{4-[(hydroxyamino)(imino)methyl]phenyl}ethyl]carbamate (2.10 g) in a microwave vial was added 4-isobutylbenzoyl chloride (1.62 g) drop-wise over a few minutes to control the exotherm. A pyridine rinse (1 mL) of the acid chloride flask was used to complete the acid chloride addition. The mixture was stirred for 1 hour and then irradiated at 120° C. for 50 min. The reaction mixture was concentrated in vacuo to ˜5 mL and then partitioned between water and dichloromethane. The organic layer was washed with brine, dried over magnesium sulfate, filtered and concentrated in vacuo to give a crude solid (4.3 g). The solid was taken up in dichloromethane and purified on a Biotage 40+M column eluting with a linear gradient of 0-100% ethyl acetate/heptanes. Column fractions containing product were combined and concentrated in vacuo to give the title compound as a solid (3.06 g)

### Step 6B. (1R)-1-{4-[5-(4-Isobutylphenyl)-1,2,4-oxadiazol-3-yl]phenyl}ethanamine

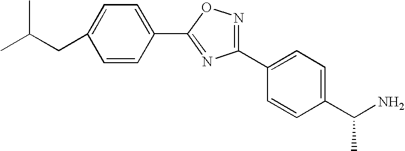

tert-Butyl [(1R)-1-{4-[5-(4-isobutylphenyl)-1,2,4-oxadiazol-3-yl]phenyl}ethyl]carbamate (3.0 gm) was dissolved in dichloromethane (60 mL) and the resulting solution cooled in an ice bath. Trifluoroacetic acid (26 mL) was added in one portion and the reaction was allowed to warm to room temperature overnight. The reaction mixture was concentrated in vacuo, then toluene was added and the mixture was again concentrated under reduced pressure. The resulting residue was slurried in ethyl ether, stirred for 0.5 hour, and then filtered. The collected solid was repeatedly washed with ethyl ether and then dried in vacuo at 40° C. The solid was slurried with warm water (50 mL) and the slurry stirred for 0.5 hour. To the slurry was added dichloromethane (40 mL) and 15% aqueous sodium hydroxide solution (3.0 mL). After separation of the layers, the organic layer was washed with 5% aqueous sodium hydroxide (10 mL) followed by brine. The aqueous layers were back-extracted with dichloromethane and the combined organic layers were dried over sodium sulfate, filtered and concentrated in vacuo to give the title compound (1.84 gm): ¹H NMR (400 MHz, DMSO-d₆) δ ppm 8.07-8.12 (2H, m), 7.99-8.05 (2H, m), 7.59 (2H, d), 7.45 (2H, d), 4.07 (1H, q), 2.57 (2H, d), 1.83-1.98 (3H, m), 1.28 (3H, d), 0.89 (6H, d).

### Preparation 7

### Ethyl cis- and trans-3-{[(1R)-1-{4-[5-(4-isobutylphenyl)-1,2,4-oxadiazol-3-yl]phenyl}ethyl]amino}cyclobutanecarboxylate

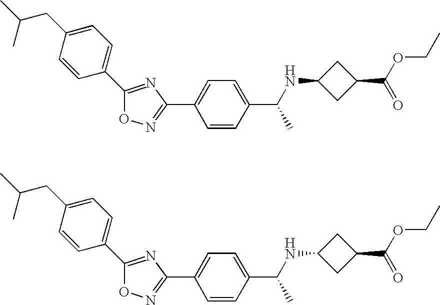

To a flask containing ethyl 3-oxocyclobutanecarboxylate (0.03 gm) was charged 2-methyltetrahydrofuran (1.5 mL) followed by (1R)-1-{4-[5-(4-isobutylphenyl)-1,2,4-oxadiazol-3-yl]phenyl}ethanamine (0.05 gm). The solution was allowed to stir at room temperature. After ˜0.5 hours, the reaction was treated with sodium triacetoxyborohydride (0.05 gm) in one portion. The resulting mixture was left to stir at room temperature. After stirring overnight, the cloudy reaction mixture was diluted with 2-methyltetrahydrofuran (20 ml) and treated with saturated aqueous sodium bicarbonate (10 mL). The mixture was vigorously stirred and then the layers were separated. The aqueous layer was extracted with 2-methyltetrahydrofuran (2×10 mL). The organic layers were combined and washed with brine, dried over sodium sulfate, filtered and concentrated under reduced pressure to give a viscous oil. The oil was purified by flash column chromatography on silica gel eluting with 15-20% 3:1 ethyl acetate:ethanol in dichloromethane. The product-containing fractions were combined and concentrate under reduced pressure to afford a viscous oil (0.06 gm) as a mixture of cis/trans isomers.

A sample of the isomeric mixture (0.2 gm) was separated using supercritical fluid chromatography on a ChiralPak AD-H (Chiral Technologies) column (30×250 mm), loaded at 20 mg/mL in ethanol (1 mL/injection), and eluting with 45% ethanol at a flow rate of 70 mL/min, to give the cis-isomer title compound (0.12 gm) and the trans-isomer title compound (0.04 gm): cis-isomer ¹H NMR (400 MHz, CDCl₃) δ ppm 8.06-8.18 (4H, m), 7.46 (2H, d), 7.33 (2H, d), 4.11 (2H, q), 3.87 (1H, q), 3.02-3.14 (1H, m), 2.60-2.69 (1H, m), 2.58 (2H, d), 2.45-2.55 (1H, m), 2.25-237 (1H, m), 1.82-2.02 (3H, m), 1.38 (3H, d), 1.24 (3H, t), 0.94 (6H, d, J=6.7 Hz); trans-isomer ¹H NMR (400 MHz, CDCl₃) δ ppm 8.07-8.16 (4H, m), 7.44 (2H, d), 7.32 (2H, d), 4.10 (2H, q), 3.79-3.88 (1H, m), 3.40-3.51 (1H, m), 2.89-3.03 (1H, m), 2.57 (2H, d), 2.45-2.54 (1H, m), 2.26-2.38 (1H, m), 1.83-2.10 (3H, m), 1.38 (3H, d), 1.22 (3H, t), 0.93 (6H, d).

### Example 1

### trans-3-{[(1R)-1-{-4-[5-(4-Isobutylphenyl)-1-1,2,4-oxadiazol-3-yl]phenyl}ethyl]amino}cyclobutanecarboxylic acid

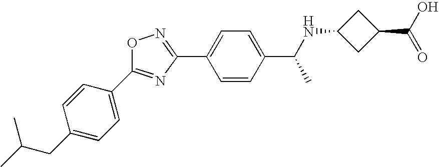

To a solution of ethyl trans-3-{[(1R)-1-{4-[5-(4-isobutylphenyl)-1,2,4-oxadiazol-3-yl]phenyl}ethyl]amino}cyclobutanecarboxylate (0.033 gm) dissolved in tetrahydrofuran (3 mL) and methanol (1 mL) at room temperature was added water (2 mL) followed by lithium hydroxide hydrate (0.031 gm). The reaction was stirred at room temperature for 2.5 hours, then concentrated under a stream of nitrogen. The residue was diluted with 1 mL of water and treated with 1 N HCl to adjust the pH to 5. The resulting solid was collected, rinsed with ˜1 mL water, air-dried, and finally dried in vacuo to give the title compound to give as a white solid (0.025 gm): MS MI-H=420; ¹H NMR (400 MHz, DMSO-d₆) δ ppm 8.10 (2H, d), 8.06 (2H, d), 7.62 (2H, d), 7.46 (2H, d), 3.85-4.05 (1H, m), 2.80-2.96 (1H, m), 2.58 (2H, d), 1.84-2.33 (6, m), 1.36 (3H, d), 0.89 (6H, d).

### Preparation 8

### 4-(2-Methyl-1,3-dioxolan-2-yl)benzonitrile

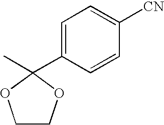

4-Cyanoacetophenone (350 g, 2.4 moles), ethylene glycol (210 g, 3.3 moles) and borontrifluoroetherate (34 g, 241 mmol) were heated at reflux in toluene (1.0 L) in a flask equipped with a Dean-Stark Trap for 6 hours. The solution was stirred 16 hours at room temperature. To the solution was added additional ethylene glycol (50 mL) and the solution was refluxed for an additional 3 hours. Boron trifluoroetherate (5 mL) was added and the solution refluxed for an additional 1 hour at which time GC/MS indicated the reaction was complete. The solution was cooled to room temperature and extracted with saturated sodium bicarbonate (2×400 mL) followed by saturated ammonium chloride (400 mL). The solution was dried over sodium sulfate, filtered and the solvent removed to afford a solid. The solid was mixed in ethyl acetate (100 mL) and heated to reflux. The resulting solution was cooled to 50° C., heptanes (500 mL) were added and the solution stirred overnight at room temperature. The resulting crystals were collected by vacuum filtration. The 4-(2-methyl-1,3-dioxolan-2-yl)benzonitrile (362 grams, 1.5 moles) was isolated as yellow crystals. (62% yield) ¹H NMR (400 MHz, DMSO-d₆) δ ppm 7.85 (d, 2H), 7.61 (d, 2H), 4.01 (m, 2H), 3.69 (m, 2H), 1.56 (s, 3H). HRMS Calcd for M+H. C₁₁H₁₂NO₂ 190.0863; Found 190.0886

### Preparation 9

### N-Hydroxy-4-(2-methyl-1,3-dioxolan-2-yl)benzenecarboximidamide

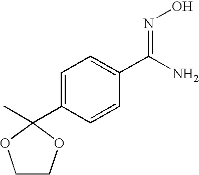

Potassium hydroxide (156.0 g, 2780 mmol) was dissolved in methanol (1500 mL) and an exotherm was noted. When the reaction mixture returned to room temperature, hydroxylamine hydrochloride (193 g, 2780 mmol) was added and the solution stirred for 15 minutes. The 4-(2-methyl-1,3-dioxolan-2-yl)benzonitrile (351 g, 1860 mmol) was added and the solution stirred for 5 minutes at room temperature. The reaction mixture was heated to 62° C. for 1.5 hours and cooled to room temperature for 16 hours. The product crystallized and was collected by vacuum filtration. The filtrate solvent was reduced to a volume of about 500 mL. A second batch of crystals was obtained and isolated by vacuum filtration. The batches were combined to give N-hydroxy-4-(2-methyl-1,3-dioxolan-2-yl)benzenecarboximidamide (358 grams, 1.61 moles) as crystals (86% yield). ¹H NMR (400 MHz, DMSO-d₆) δ ppm 9.64 (s, 1H) 9.65 (d, 2H), 7.41 (d, 2H), 5.81 (bs, 2H), 3.99 (m, 2H), 3.69 (m, 2H), 1.56 (s, 3H). HRMS Calcd for M+H, C₁₁H₁₅N₂O₃ 223.1077; Found 223.1070.

### Preparation 10

### 1-{4-[5-(4-Isobutylphenyl)-1,2,4-oxadiazol-3-yl]phenyl}ethanone

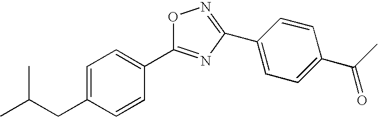

4-iso-Butylbenzoic acid (126 g) and carbonyldiimidazole (131.0 g) were mixed together in 1-methyl-2-pyrrolidinone (200 mL). Gas evolution was noted and the mixture stirred at room temperature for 15 minutes. The solution became homogeneous and was stirred for 3 hours. The N-hydroxy-4-(2-methyl-1,3-dioxolan-2-yl)benzenecarboximidamide (150 g, 674 mmol) was added and the solution was stirred for 1 hour and the reaction had became a thick heterogeneous mixture. The mixture was heated to 107° C. and became homogeneous. The solution was kept at 107° C. for 2 h. The solution was cooled to room temperature and water (800 mL) was added. Solids formed in the solution and the mixture was stirred at room temperature for 16 hours. The solids were collected by vacuum filtration. The solids were mixed in methanol (700 mL) and 2.0 M aqueous hydrochloric acid was then added (50 mL). The solution was heated to 60° C. for 1 hour. The reaction was complete and crystals began to form. The mixture was cooled to room temperature for 16 hours. The crystals were collected. 1-{4-[5-(4-isobutylphenyl)-1,2,4-oxadiazol-3-yl]phenyl}ethanone (156 g) was obtained as light yellow crystals (69° A yield). ¹H NMR (400 MHz, DMSO-d₆) δ ppm 8.24 (d, 2H), 8.17 (d, 2H), 8.13 (d, 2H), 7.47 (d, 2H), 2.66 (s, 3H), 2.59 (d, 2H), 1.90 (m, 1H), 0.90 (d, 6H). HRMS Calcd for M+H, C₂₀H₂₁N₂O₂ 321.1598; Found 321.1624.

### Preparation 11

### Ethyl cis-3-{[(1R)-1-{-4-[5-(4-isobutylphenyl)-1,2,4-oxadiazol-3-yl]phenyl}ethyl]amino}cyclobutanecarboxylate

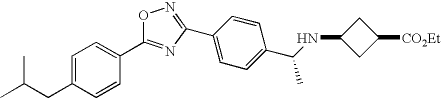

Ethyl cis-3-aminocyclobutylcarboxylate hydrochloride (20.2 g) was mixed in tetrahydrofuran (600 mL). Triethylamine (13.3 g, 131 mmol) was added and the solution stirred for 1 hour. The mixture was stirred and titanium ethoxide (25.0 mL) and 1-{4-[5-(4-isobutylphenyl)-1,2,4-oxadiazol-3-yl]phenyl}ethanone (30.0 g) were added. The solution was stirred for 3 hours at room temperature after which additional titanium ethoxide (15 mL) was added. To the solution was added additional amine (7.2 grams, 40.2 mmol) and triethylamine (7.21 g). The solution was mixed for 1 h at room temperature. Sodium borohydride (15 grams) was added and the solution was stirred for 16 hours. 2.0 M Aqueous ammonium hydroxide (200 mL) was added and the mixture was stirred for 1 h. The precipitates were removed by vacuum filtration through Celite. The filter cake was collected, stirred with ethyl acetate (300 mL) and solvent removed at reduced pressure. This filter cake washing was repeated 2 additional times with ethyl acetate. The filtrates were combined, washed with saturated aqueous sodium bicarbonate (2×200 mL) followed by brine (200 mL). The aqueous layers were combined and back extracted with ethyl acetate (200 mL). The combined organic solutions were dried over sodium sulfate and solvent removed at reduced pressure. 33.4 grams of a light yellow oil was obtained. The product was isolated by silica gel chromatography (Biotage 75 L, 30-50% ethyl acetate in heptanes). Ethyl cis-3-{[1-{4-[5-(4-isobutylphenyl)-1,2,4-oxadiazol-3-yl]phenyl}ethyl]amino}cyclobutanecarboxylate (23.0 g, 51.4 mmol) was obtained as a light yellow oil (45.5% yield).

A sample of the enantiomeric mixture (41.5 g) was separated using supercritical fluid chromatography on a ChiralPak AD-H (Chiral Technologies) column (30×250 mm). Elution with 50% ethanol/carbon dioxide at a flow rate of 70 mL/min led to isolation of the title compound (9.3 g): [λ]_{D}²⁵=+31.6 (c=1, MeOH); ¹H NMR (400 MHz, DMSO-d₆) δ ppm 8.10 (d, 2H), 8.02 (d, 2H), 7.51 (d, 2H), 7.46 (d, 2H), 4.15 (q, 2H), 3.77 (m, 1H), 2.90 (m, 1H), 2.58 (m, 3H), 2.31, (m, 1H), 2.06 (m, 1H), 1.74-1.93 (m, 3H), 1.25 (d, 3H) 1.15 (t, 3H), 0.89 (d, 6H). HRMS Calcd for M+H, C₂₇H₃₄N₃O₃ 448.2595; Found 448.2578.

### Preparation 12

### Ethyl cis-3-{[(1S)-1-{4-[5-(4-isobutylphenyl)-1,2,4-oxadiazol-3-yl]phenyl}ethyl]amino}cyclobutanecarboxylate

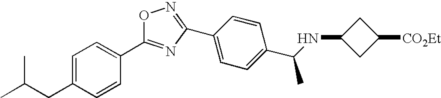

The title compound (14.2 g) was isolated from the chiral separation described above for ethyl cis-3-{[(1R)-1-{4-[5-(4-isobutylphenyl)-1,2,4-oxadiazol-3-yl]phenyl}ethyl]amino}cyclobutanecarboxylate. [λ]_{D}²⁵=−21.6 (c=1, MeOH); (400 MHz, DMSO-d₆) δ ppm (400 MHz, DMSO-d₆) 8.04 (d, 2H), 7.95 (d, 2H), 7.47 (d, 2H), 7.39 (d, 2H), 4.01-3.92 (m, 3H), 3.70 (q, 1H), 2.84 (m, 1H), 2.30-2.21 (m, 2H), 2.05-1.95, (m, 2H), 1.67-1.88 (m, 3H), 1.18 (d, 3H), 1.12-1.06 (m, 4H), 0.83 (d, 6H).

### Example 2

### cis-3-{[(1R)-1-{4-[5-(4-Isobutylphenyl)-1,2,4-oxadiazol-3-yl]phenyl}ethyl]amino}cyclobutanecarboxylic acid

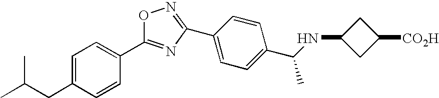

A solution of the ethyl cis-3-{[(1R)-1-{4-[5-(4-isobutylphenyl)-1,2,4-oxadiazol-3-yl]phenyl}ethyl]amino}cyclobutanecarboxylate (21.0 grams) in dioxane (150 mL) was added to a solution of water (50 mL) with potassium hydroxide (6.63 g, 118 mmol) at room temperature. The solution was heated to 50° C. for 30 min. The solution became turbid and was cooled to 35° C. 6N HCl was added dropwise and solids began to form at pH=9 (pH probe was used to monitor pH). The solution was mixed well and 6 N HCl added until the pH=6.5. The thick white solution was mixed for 1 hour at room temperature and the solids collected. The pasty solids were dried at room temperature and reduce pressure overnight. cis-3-{[(1R)-1-{-4-[5-(4-Isobutylphenyl)-1,2,4-oxadiazol-3-yl]phenyl}ethyl]amino}cyclobutanecarboxylic acid (18.9 grams, 41.5 mmol) was isolated as a white solid. (96% yield). [λ]_{D}²⁵=+8.8 (c=1, DMSO); ¹H NMR (400 MHz, DMSO-d₆) δ ppm (400 MHz, DMSO-d₆) 8.12 (d, 2H), 8.03 (d, 2H), 7.55 (d, 2H), 7.47 (d, 2H), 3.78 (m, 1H), 2.89 (m, 1H), 2.59 (m, 3H), 2.33, (m, 1H), 2.13 (m, 1H), 1.74-1.92 (m, 3H), 1.26 (d, 3H), 0.90 (d, 6H). HRMS Calcd for M+H, C₂₅H₃₀N₃O₃ 420.2282; Found 420.2302.

### Example 3

### cis-3-{[(1S)-1-{4-[5-(4-isobutylphenyl)-1,2,4-oxadiazol-3-yl]phenyl}ethyl]amino}cyclobutanecarboxylic acid, hydrochloride salt

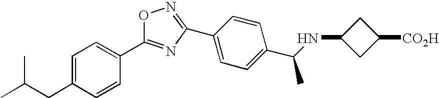

To a solution of the ethyl cis-3-{[(1S)-1-{-4-[5-(4-isobutylphenyl)-1,2,4-oxadiazol-3-yl]phenyl}ethyl]amino}cyclobutanecarboxylate (361 mg) in dioxane (10 mL) was added a 1N solution of aqueous sodium hydroxide (3.5 mL). The mixture was stirred for 1.5 hours at room temperature at which point no starting material remained. The reaction was further diluted with water (3.5 mL) and then neutralized by slow addition of an aqueous 2N hydrochloric acid solution (1.75 mL) to achieve a pH of 4-5. A white precipitate results which was filtered, washed with ice cold water, and dried. The resulting solid was slurried in a 1:1 mixture of acetonitrile-water (4 mL). A solution of aqueous 2N hydrochloric acid was added until the pH of the reaction mixture was 2. The solution was then concentrated at room temperature to remove the acetonitrile and the remaining solution was lyophilized to give a white solid. The residue was slurried in diethyl ether and filtered to give the title compound (298 mg) after drying. [λ]_{D}²⁵=−14.3. (c=1, DMSO); ¹H NMR (400 MHz, DMSO-d₆) δ ppm (400 MHz, DMSO-d₆) 8.10 (d, 2H), 8.05 (d, 2H), 7.69 (d, 2H), 7.41 (d, 2H), 4.4-4.3 (m, 1H), 2.75 (m, 1H), 2.53 (d, 2H), 2.36-2.23 (m, 3H), 2.15-1.98 (m, 2H), 1.91-1.80 (m, 1H), 1.50 (d, 3H), 0.84 (d, 6H)

1. A compound of Formula I: 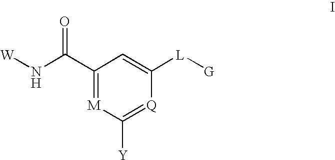
wherein
G is (C₁₋₃ alkylenyl)-R^{a} or R^{a}, wherein said C₁₋₃ alkylenyl may be substituted by one or more substituents selected from OH and F;
R^{a} is C₁₋₆ alkyl, 3- to 7-membered heterocycloalkyl, or 3- to 8-membered cycloalkyl, wherein said C₁₋₆ alkyl may be substituted by one or more substituents selected from F, —OR³⁴, —SR³⁴, —C(═O)NR³⁴R³⁵, —NR³⁴R³⁵, —NR³⁴C(═O)R³⁵, or —NR³⁴SO₂R³⁶, and wherein said heterocycloalkyl and cycloalkyl may be substituted by one or more substituents selected from R², R³, R⁴, R⁵, R¹⁰, R¹¹, R²¹;
M is N or C—R^{b};
Q is N or C—R^{c}, provided that if Q is C—R^{c}, then M is N;
W is phenyl-(C₁₋₆ alkylenyl), pyridyl-(C₁₋₆ alkylenyl), or 9-membered heteroaryl-(C₁₋₆-alkylenyl), wherein said phenyl, pyridyl, or 9-membered heteroaryl is substituted by one or more groups selected from R³⁰ and R³¹;
Y is C₁₋₆ alkyl, C₁₋₆ haloalkyl; or C₁₋₆ hydroxyalkyl;
L is 5-membered heteroaryl;
R² is H, F, CN, —OR⁵, R¹², —C(═O)R⁷, —NR⁸R³³, —NR⁸C(═O)R⁹, —NR⁸SO₂R³⁶, or —SO₂R¹²;
R³ is H, F, CN, or —OR²²;
R⁴ is H, —(C₁₋₆ alkylene)R⁶, —C(═O)R⁹, or —SO₂R¹²;
R⁵ is H or —(C₁₋₆ alkyl), wherein said C₁₋₆ alkyl may be substituted by one or more R²⁶ substituents;
R⁶ is H, CN, —OR²³, —SO₂R³⁷, —NR²⁴C(═O)R²³, —NR²⁴SO₂R³⁷, or 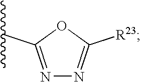
R⁷ is —(C₁₋₆ alkyl), —(C₁₋₆ haloalkyl), —(C₁₋₆ alkylene)OH, —NR³⁸R³⁹, —NHSO₂CH₃, or —OR²⁵;
R⁹ is —(C₁₋₆ alkylene)R²⁸, —NHR²⁴, or —OR²⁵;
R¹⁰ is H, F, CN, R¹², or —C(═O)R⁷;
R¹¹ is H, F, CN, —OR⁵, R¹²—NR⁸R³³, —NR⁸C(═O)R⁹, —NR⁸SO₂R³⁶, or —SO₂R¹²;
R¹² is —(C₁₋₆ alkyl), wherein said C₁₋₆ alkyl may be substituted by one or more substituents selected from CN, —OR²³, —SR²³, —SO₂R³⁷, —NR⁸R³³, —NR²⁴C(═O)R²³, and —NR²⁴SO₂R²³, provided that any one carbon atom of said C₁₋₆ alkyl is not substituted by more than one CN or more than one —OR²³;
R^{b}, R^{c}, R⁸, R²⁰, R²², R²⁴, R²⁵, R³³, R³⁴, and R³⁵ are independently H or —(C₁₋₆ alkyl);
R²¹ is H, F, or R¹²;
R²³ is H, —(C₁₋₆ alkyl), or —(C₁₋₆ alkylene)OH;
R²⁶ is H, OH, halo, NH₂, or SH;
R²⁸ is H, NH₂, or —OR²⁹;
R²⁹ is H, (C₁₋₆ alkyl), or —C(═O)(C₁₋₆ alkyl);
R³⁰ is H or F;
R³¹ is F, Cl, Br, —CN, —C(═O)NH₂, —OH, —OR³², —OCH₂CH₂OR²⁵, R³², or (C₁₋₆ alkyl) optionally substituted by —OR²⁵ or —OCH₂CH₂OR²⁵
where R³⁰ and R³¹ when adjacent may be taken together to constitute a group of the formula —O—(CH₂)ₙ— or —O—(CH₂)ₙ—O—;
n is 1 or 2;
R³² is —(C₁₋₆ alkyl) optionally substituted with one, two, or three F;
R³⁶ is —(C₁₋₆ alkyl) or —(C₃₋₆ cycloalkyl); and
R³⁷ is —(C₁₋₆ alkyl), —(C₃₋₆ cycloalkyl), or —(C₁₋₆ alkylene)OH;
R³⁸ and R³⁹ are independently H, —(C₁₋₆ alkyl), or R³⁸ and R³⁹ taken together form a 5- or 6-membered heterocycloalkyl;
or a pharmaceutically-acceptable salt thereof.

2. A compound according to claim 1 wherein R^{a} is C₁₋₆ alkyl, 1,4-dioxanyl, piperidinyl, cyclohexyl, morpholinyl, thiomorpholinyl, tetrahydropyranyl, tetrahydropyranonyl, pyrrolidinyl, cyclopentyl, 7-oxa-bicyclo[2.2.1]heptanyl, piperazinyl, 1,1′-dioxothiomorpholinyl, tetrahydro-1,1′-dioxothiopyranyl, tetrahydrothiopyranyl, piperidinonyl, tetrahydrofuryl, pyrrolidinonyl, or oxazolidinonyl, wherein said alkyl may be substituted by one or more substituents selected from —OR³⁴, —SR³⁴, —C(═O)NR³⁴R³⁵, —NR³⁴R³⁵, —NR³⁴C(═O)R³⁵, or —NR³⁴SO₂R³⁵, and wherein said 1,4-dioxyl, piperidinyl, cyclohexyl, morpholinyl, thiomorpholinyl, tetrahydropyranyl, tetrahydropyranonyl, pyrrolidinyl, cyclopentyl, 7-oxa-bicyclo[2.2.1]heptanyl, piperazinyl, 1,1′-dioxothiomorpholinyl, tetrahydro-1,1′-dioxothiopyranyl, tetrahydrothiopyranyl, piperidinonyl, tetrahydrofuryl, pyrrolidinonyl, or oxazolidinonyl may be R⁴, R⁵, R¹⁰, substituted by one or more substituents selected from R², R³, R⁴, R⁵, R¹⁰, R¹¹, and R²¹.

3. A compound according to claim 1 wherein R^{a} is C₁₋₆ alkyl, 1,4-dioxanyl substituted by R¹⁰ and R²¹, piperidinyl substituted by R² and R³ or by R⁴, cyclohexyl substituted by R² and R³ or by R⁴, morpholinyl substituted by R⁴, thiomorpholinyl substituted by R¹⁰ and R²¹, tetrahydropyranyl substituted by R³ and R¹¹ or by R¹⁰ and R²¹, tetrahydropyranonyl, pyrrolidinyl substituted by R² and R³ or by R⁴, cyclopentyl substituted by R² and R³, 7-oxa-bicyclo[2.2.1]heptanyl, piperazinyl substituted by R⁴ and R⁵, 1,1′-dioxothiomorpholinyl, tetrahydro-1,1′-dioxothiopyranyl, tetrahydrothiopyranyl, piperidinonyl substituted by R⁵, tetrahydrofuryl substituted by R³ and R¹¹, pyrrolidinonyl substituted by R⁵, oxazolidinonyl substituted by R⁵, wherein said alkyl may be substituted by one or more substituents selected from —OR³⁴, —SR³⁴, —C(═O)NR³⁴R³⁵, —NR³⁴R³⁵, —NR³⁴C(═O)R³⁵, or —NR³⁴SO₂R³⁵.

4. A compound according to claim 3 wherein R^{a} is C₁₋₆ alkyl, 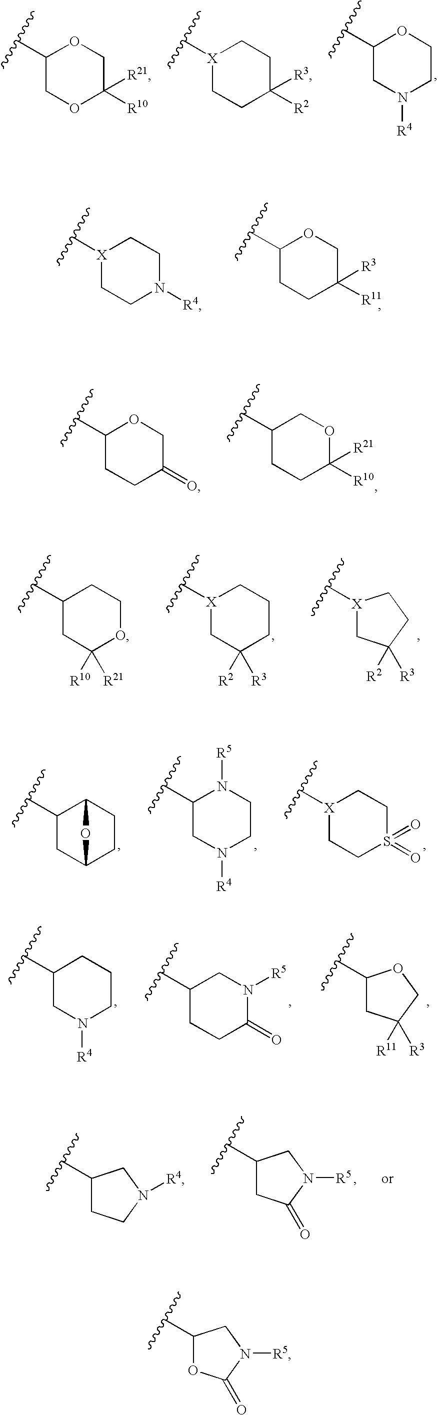
wherein said alkyl may be substituted by one or more substituents selected from —OR³⁴, —SR³⁴, —C(═O)NR³⁴R³⁵, —NR³⁴R³⁵, —NR³⁴C(═O)R³⁵, —NR³⁴SO₂R³⁵; and
X is N or CH, provided that when X is N, G is (C₂₋₃ alkylenyl)-R^{a}, wherein said C₁₋₃ alkylenyl may be substituted by one or more substituents selected from OH and F.

5. A compound according to claim 4 wherein R^{a} is C₁₋₆ alkyl, 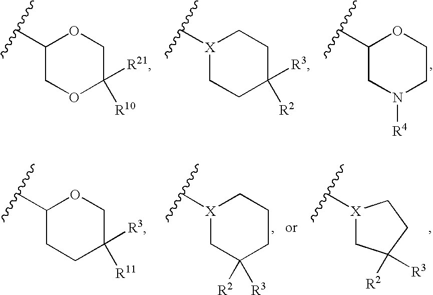
wherein said alkyl may be substituted by one or more substituents selected from —OR³⁴, —SR³⁴, —C(═O)NR³⁴R³⁵, —NR³⁴R³⁵, —NR³⁴C(═O)R³⁵, or —NR³⁴SO₂R³⁵.

6. A compound according to claim 5 wherein R^{a} is C₁₋₆ alkyl, wherein said alkyl may be substituted by one or more substituents selected from —OR³⁴, —SR³⁴, —C(═O)NR³⁴R³⁵, —NR³⁴R³⁵, —NR³⁴C(═O)R³⁵, or —NR³⁴SO₂R³⁵.

7. A compound according to claim 6 wherein R^{a} is C₁₋₆ alkyl, wherein said alkyl may be substituted by one or more —OR³⁴.

8. A compound according to claim 5 wherein R^{a} is

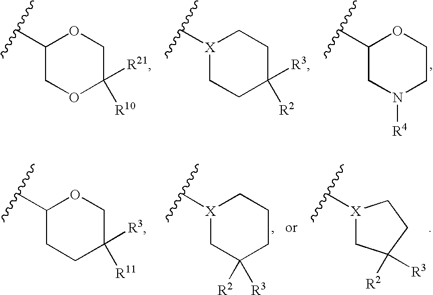

9. A compound according to claim 8 wherein R^{a} is

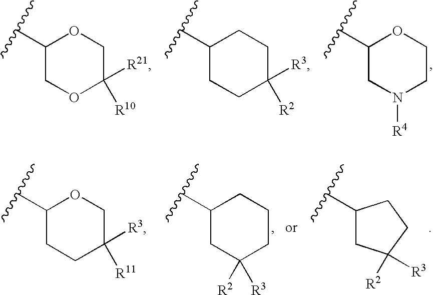

10. A compound according to claim 1 wherein:
G is —CH₂R^{a}, —CH₂CH₂R^{a}, —CH(R^{a})CH₃, —CH₂CH₂CH₂R^{a}, —CH₂CH(CH₃)R^{a}, —CH(R^{a})CH₂CH₃, or R^{a}, wherein said CH, CH₂, and CH₃ groups may be substituted by one or more substituents selected from OH and F;
R^{a} is methyl, ethyl, 1-propyl, 2-propyl, 1-butyl, 2-butyl, 2,2-dimethylethyl, 1-pentyl, 2-pentyl, 2,2-dimethylpropyl, 1-hexyl, aziridinyl, azetidinyl, pyrrolidinyl, pyrazolidinyl, imidazolidinyl, piperidinyl, piperazinyl, oxiranyl, oxetanyl, tetrahydrofuryl, dioxolyl, tetrahydropyranyl, dioxanyl, oxazetidinyl, isoxazolidinyl, oxazolidinyl, morpholinyl, oxazinanyl, cyclopropyl, cyclobutyl, cyclopentyl, or cyclohexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by one or more substituents selected from ═OR³⁴, —SR³⁴, —C(═O)NR³⁴R³⁵, —NR³⁴R³⁵, —NR³⁴C(═O)R³⁵, or —NR³⁴SO₂R³⁶, and wherein said aziridinyl, azetidinyl, pyrazolidinyl, imidazolidinyl, piperidinyl, piperazinyl, oxiranyl, oxetanyl, tetrahydrofuryl, dioxolyl, tetrahydropyranyl, dioxanyl, oxazetidinyl, isoxazolidinyl, oxazolidinyl, morpholinyl, oxazinanyl, cyclopropyl, cyclobutyl, cyclopentyl, or cyclohexyl may be substituted by one or more substituents selected from R², R³, R⁴, R⁵, R¹⁰, R¹¹, and R²¹;
W is phenyl-(C₁₋₆ alkylenyl), pyridyl-(C₁₋₆ alkylenyl), or indolyl-(C₁₋₆ alkylenyl), wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene, and wherein said phenyl, pyridyl, or indolyl is substituted by one or more groups selected from R³⁰ and R³¹;
Y is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein Y may be substituted by OH or by one or more substituents selected from F, Cl, and Br;
L is triazolyl, tetrazolyl, or oxadiazolyl;
R⁴ is H, —(C₁₋₆ alkylene)R⁶, —C(═O)R⁹, or —SO₂R¹², wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene, and wherein said phenyl, pyridyl, or indolyl is substituted by one or more groups selected from R³⁰ and R³¹;
R⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl, may be substituted by one or more R²⁶ substituents;
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —NHR²⁴, or —OR²⁵, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH or by one or more substituents selected from F, Cl, and Br;
R⁹ is —(C₁₋₆ alkylene)R²⁸, —NHR²⁴, or —OR²⁵;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be substituted by one or more substituents selected from CN, —OR²³, —SO₂R³⁷, —NR⁸R³³, —NR²⁴C(═O)R²³, and —NR²⁴SO₂R²³, provided that any one carbon atom of R¹² is not substituted by more than one CN or more than one —OR²³;
R^{b}, R^{c}, R⁸, R²⁰, R²², R²⁴, R²⁵, R³³, R³⁴, and R³⁵ are independently H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, 1-hexyl, may be substituted by OH;
R²⁶ is H, OH, F, Cl, Br, NH₂, or SH;
R²⁹ is H, —C(═O)CH₃, —C(═O)CH₂CH₃, —C(═O)(CH₂)₂CH₃, —C(═O)CH(CH₃)₂, —C(═O)(CH₂)₃CH₃, —C(═O)CH(CH₃)CH₂CH₃, —C(═O)CH₂CH(CH₃)₂, —C(═O)(CH₂)₄CH₃, —C(═O)CH₂CH(CH₃)CH₂CH₃, —C(═O)CH₂C(CH₃)₃, or —C(═O)(CH₂)₅CH₃;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —OCH₂CH₂OR²⁵;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² may be substituted with one, two, or three F;
R³⁶ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁷ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, 1-hexyl, may be substituted by OH;
or a pharmaceutically-acceptable salt thereof.

11. A compound according to claim 1 having Formula II:

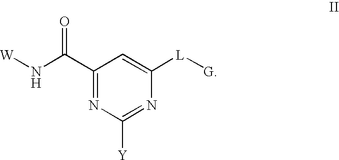

12. A compound according to claim 11 having Formula IIA:

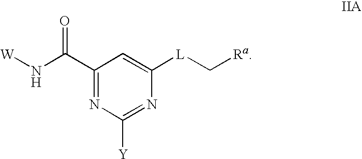

13. A compound according to claim 12 wherein L is triazolyl, tetrazolyl, or oxadiazolyl.

14. A compound according to claim 13 wherein L is

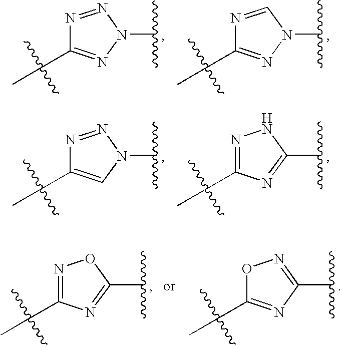

15. A compound according to claim 14 wherein L is

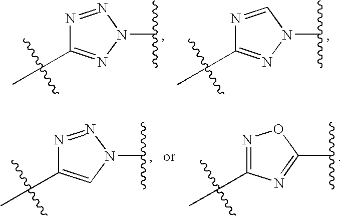

16. A compound according to claim 14 of Formula IIA-1

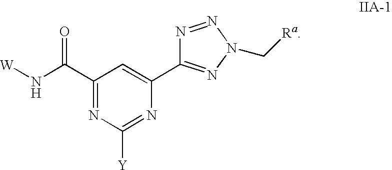

17. A compound according to claim 14 of Formula IIA-2

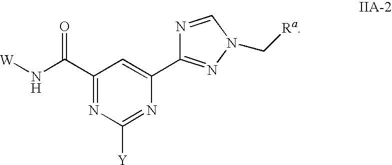

18. A compound according to claim 14 of Formula IIA-3

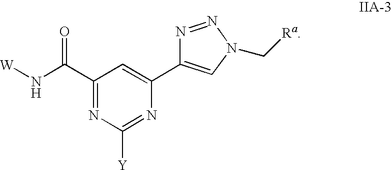

19. A compound according to claim 14 of Formula IIA-4

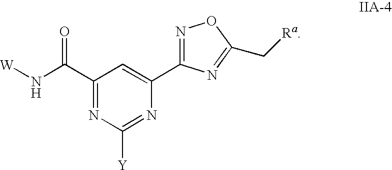

20. A compound according to claim 15
wherein R^{a} is C₁₋₆ alkyl, 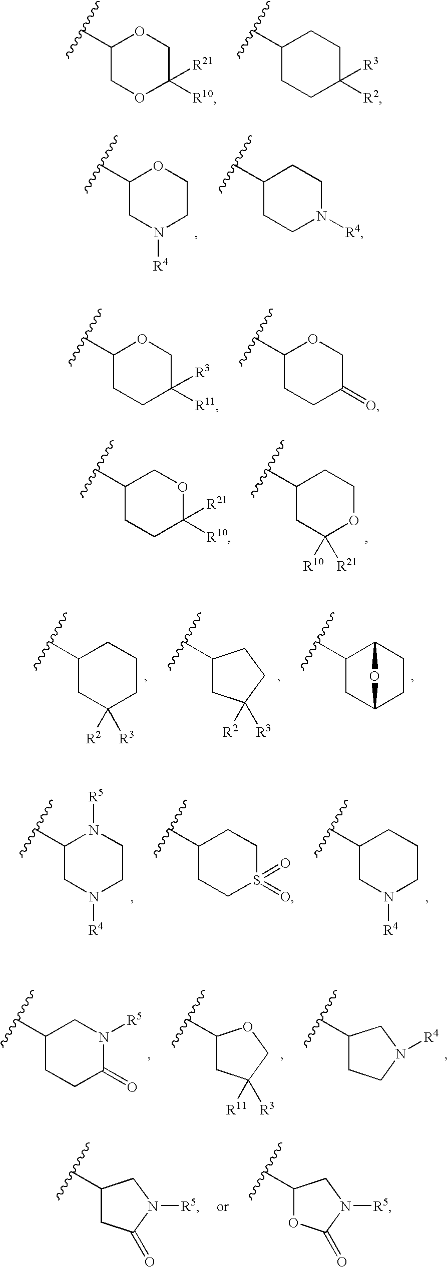
wherein said alkyl may be substituted by one or more substituents selected from ═OR³⁴, —SR³⁴, —C(═O)NR³⁴R³⁵, —NR³⁴R³⁵, —NR³⁴C(═O)R³⁵, or —NR³⁴SO₂R³⁵.

21. A compound according to claim 20 wherein R^{a} is C₁₋₆ alkyl, 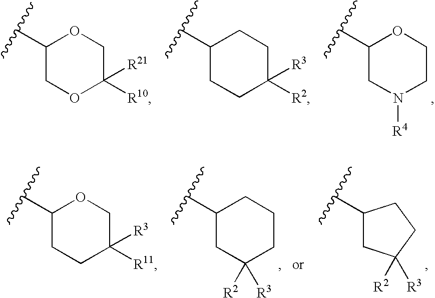
wherein said alkyl may be substituted by one or more substituents selected from —OR³⁴, —SR³⁴, —C(═O)NR³⁴R³⁵, —NR³⁴R³⁵, —NR³⁴C(═O)R³⁵, or —NR³⁴SO₂R³⁵.

22. A compound according to claim 20 wherein R^{a} is C₁₋₆ alkyl, wherein said alkyl may be substituted by one or more substituents selected from —OR³⁴, —SR³⁴, —C(═O)NR³⁴R³⁵, —NR³⁴R³⁵, —NR³⁴C(═O)R³⁵, or —NR³⁴SO₂R³⁵.

23. A compound according to claim 22 wherein R^{a} is C₁₋₆ alkyl, wherein said alkyl may be substituted by one or more —OR³⁴.

24. A compound according to claim 21 wherein R^{a} is

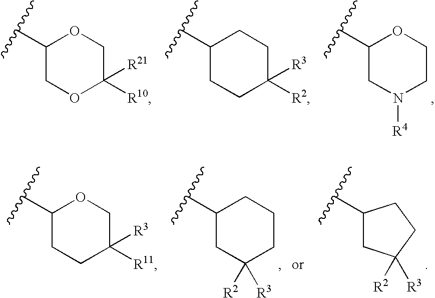

25. A compound according to claim 15 wherein:
R^{a} is methyl, ethyl, 1-propyl, 2-propyl, 1-butyl, 2-butyl, 2,2-dimethylethyl, 1-pentyl, 2-pentyl, 2,2-dimethylpropyl, 1-hexyl, aziridinyl, azetidinyl, pyrrolidinyl, pyrazolidinyl, imidazolidinyl, piperidinyl, piperazinyl, oxiranyl, oxetanyl, tetrahydrofuryl, dioxolyl, tetrahydropyranyl, dioxanyl, oxazetidinyl, isoxazolidinyl, oxazolidinyl, morpholinyl, oxazinanyl, cyclopropyl, cyclobutyl, cyclopentyl, or cyclohexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by one or more substituents selected from ═OR³⁴, —SR³⁴, —C(═O)NR³⁴R³⁵, —NR³⁴R³⁵, —NR³⁴C(═O)R³⁵, or —NR³⁴SO₂R³⁶, and wherein said aziridinyl, azetidinyl, pyrrolidinyl, pyrazolidinyl, imidazolidinyl, piperidinyl, piperazinyl, oxiranyl, oxetanyl, tetrahydrofuryl, dioxolyl, tetrahydropyranyl, dioxanyl, oxazetidinyl, isoxazolidinyl, oxazolidinyl, morpholinyl, oxazinanyl, cyclopropyl, cyclobutyl, cyclopentyl, or cyclohexyl may be substituted by one or more substituents selected from R², R³, R⁴, R⁵, R¹⁰, R¹¹, and R²¹;
W is phenyl-(C₁₋₆ alkylenyl), pyridyl-(C₁₋₆ alkylenyl), or indolyl-(C₁₋₆ alkylenyl), wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene, and wherein said phenyl, pyridyl, or indolyl is substituted by one or more groups selected from R³⁰ and R³¹;
Y is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein Y may be substituted by OH or by one or more substituents selected from F, Cl, and Br;
L is triazolyl, tetrazolyl, or oxadiazolyl;
R⁴ is H, —(C₁₋₆ alkylene)R⁶, —C(═O)R⁹, or —SO₂R¹², wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene, and wherein said phenyl, pyridyl, or indolyl is substituted by one or more groups selected from R³⁰ and R³¹;
R⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl, may be substituted by one or more R²⁶ substituents;
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —NHR²⁴, or —OR²⁵, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH or by one or more substituents selected from F, Cl, and Br;
R⁹ is —(C₁₋₆ alkylene)R²⁸, —NHR²⁴, or —OR²⁵;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be substituted by one or more substituents selected from CN, —OR²³, —SO₂R³⁷, —NR⁸R³³, —NR²⁴C(═O)R²³, and —NR²⁴SO₂R²³, provided that any one carbon atom of R¹² is not substituted by more than one CN or more than one —OR²³;
R^{b}, R^{c}, R⁸, R²⁰, R²², R²⁴, R²⁵, R³³, R³⁴, and R³⁵ are independently H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, 1-hexyl, may be substituted by OH;
R²⁶ is H, OH, F, Cl, Br, NH₂, or SH;
R²⁹ is H, —C(═O)CH₃, —C(═O)CH₂CH₃, —C(═O)(CH₂)₂CH₃, —C(═O)CH(CH₃)₂, —C(═O)(CH₂)₃CH₃, —C(═O)CH(CH₃)CH₂CH₃, —C(═O)CH₂CH(CH₃)₂, —C(═O)(CH₂)₄CH₃, —C(═O)CH₂CH(CH₃)CH₂CH₃, —C(═O)CH₂C(CH₃)₃, or —C(═O)(CH₂)₅CH₃;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —OCH₂CH₂OR²⁵;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² may be substituted with one, two, or three F;
R³⁶ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁷ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, 1-hexyl, may be substituted by OH;
or a pharmaceutically-acceptable salt thereof.

26. A compound according to claim 15 wherein Y is methyl.

27. A compound according to any one of claims claim 15 wherein W is phenyl-(C₁₋₆ alkylenyl), wherein said phenyl is substituted by one or more groups selected from R³⁰ and R³¹.

28. A compound according to claim 27 wherein W is phenylmethyl, wherein said phenyl is substituted by one or more groups selected from R³⁰ and R³¹.

29. A compound having the structure:

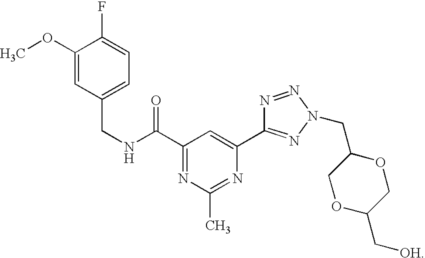

30. A compound having the structure:

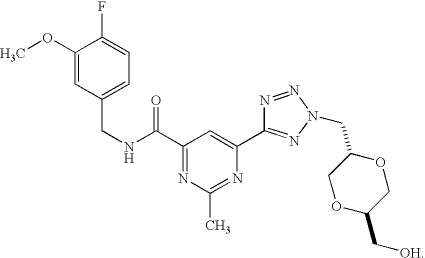

31. A compound having the structure:

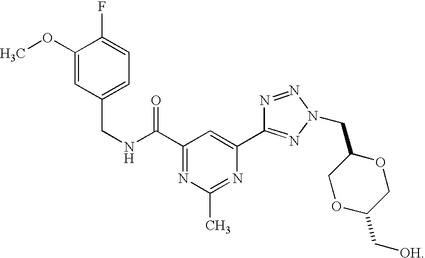

32. A compound having the structure:

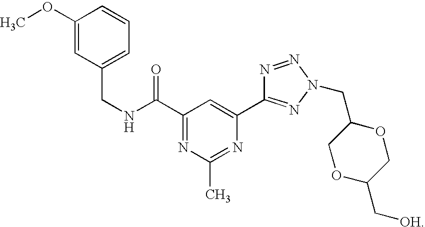

33. A compound having the structure:

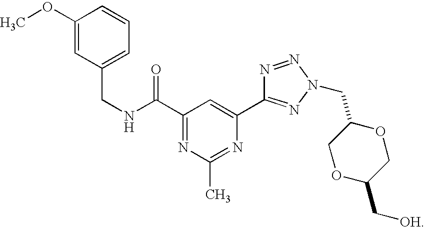

34. A compound having the structure:

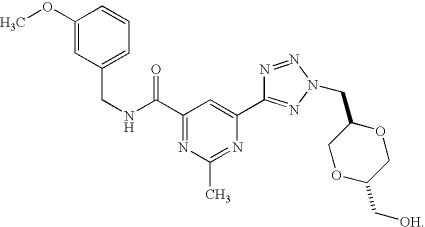

35. A compound having the structure:

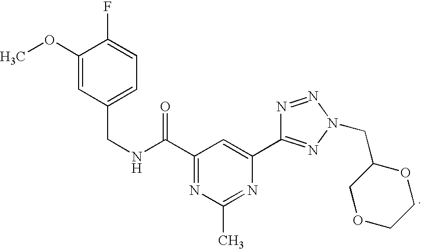

36. A compound having the structure:

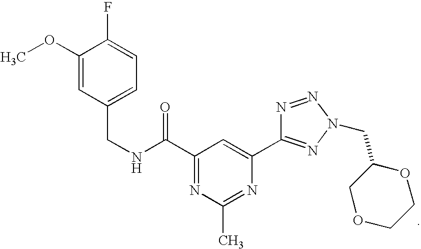

37. A compound having the structure:

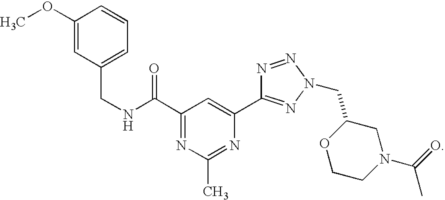

38. A compound having the structure:

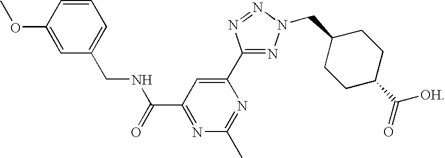

39. A compound having the structure:

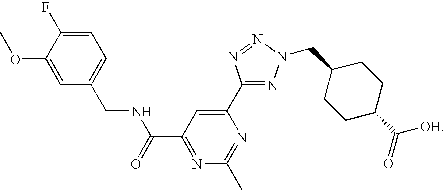

40. A compound according to claim 1 having Formula III:

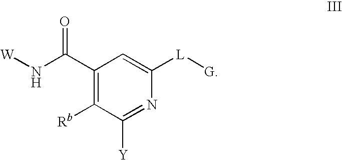

41. A compound according to claim 40 having Formula IIIA:

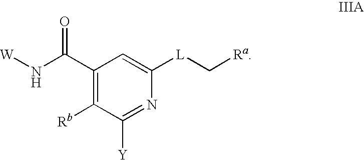

42. A compound according to claim 41 wherein L is triazolyl, tetrazolyl, or oxadiazolyl.

43. A compound according to claim 42 wherein L is

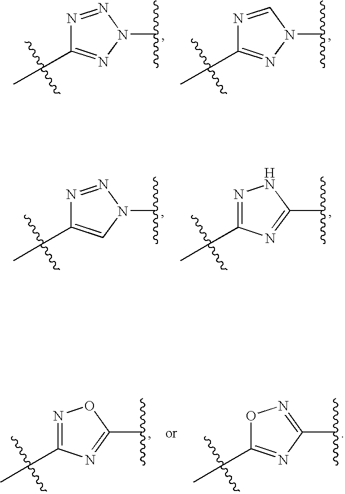

44. A compound according to claim 43 wherein L is

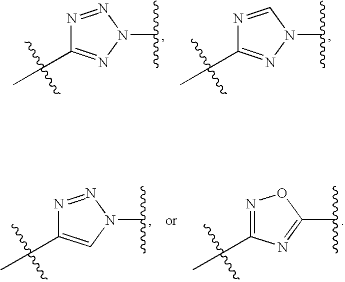

45. A compound according to claim 41 of Formula IIIA-1

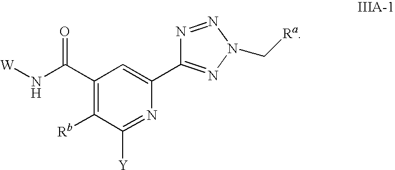

46. A compound according to claim 41 of Formula IIIA-2

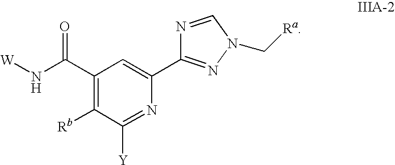

47. A compound according to claim 41 of Formula IIIA-3

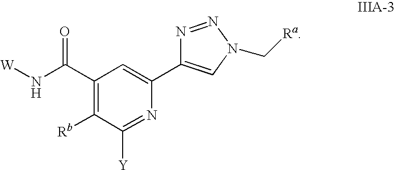

48. A compound according to claim 41 of Formula IIIA-4

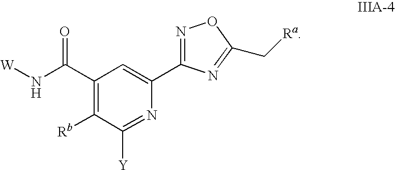

49. A compound according to claim 43 wherein R^{a} is C₁₋₆ alkyl, 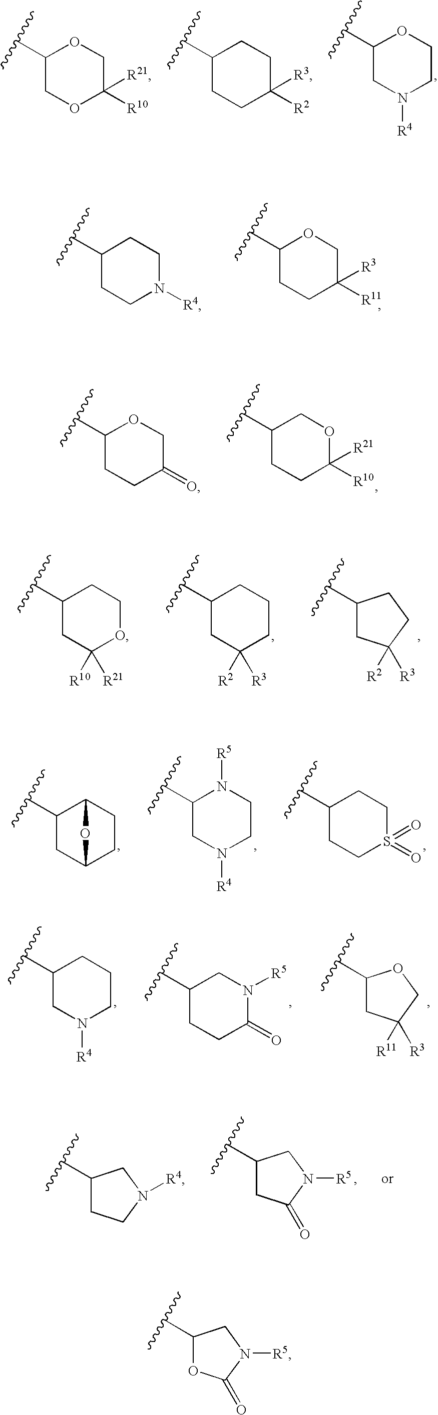
where in said alkyl may be substituted by one or more substituents selected from —OR³⁴, —SR³⁴, —C(═O)NR³⁴R³⁵, —NR³⁴R³⁵, —NR³⁴C(═O)R³⁵, or —NR³⁴SO₂R³⁵.

50. A compound according to claim 49 wherein R^{a} is C₁₋₆ alkyl, 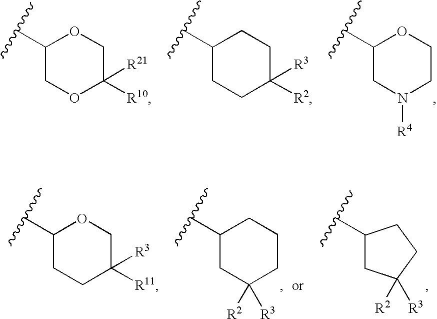
wherein said alkyl may be substituted by one or more substituents selected from —OR³⁴, —SR³⁴, —C(═O)NR³⁴R³⁵, —NR³⁴R³⁵, —NR³⁴C(═O)R³⁵, or —NR³⁴SO₂R³⁵.

51. A compound according to claim 49 wherein R^{a} is C₁₋₆ alkyl, wherein said alkyl may be substituted by one or more substituents selected from —OR³⁴, —SR³⁴, —C(═O)NR³⁴R³⁵, —NR³⁴R³⁵, —NR³⁴C(═O)R³⁵, or —NR³⁴SO₂R³⁵.

52. A compound according to claim 51 wherein R^{a} is C₁₋₆ alkyl, wherein said alkyl may be substituted by one or more —OR³⁴;

53. A compound according to claim 50 wherein R^{a} is

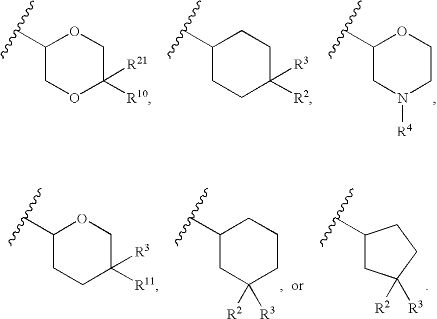

54. A compound according to claim 44 wherein:
R^{a} is methyl, ethyl, 1-propyl, 2-propyl, 1-butyl, 2-butyl, 2,2-dimethylethyl, 1-pentyl, 2-pentyl, 2,2-dimethylpropyl, 1-hexyl, aziridinyl, azetidinyl, pyrrolidinyl, pyrazolidinyl, imidazolidinyl, piperidinyl, piperazinyl, oxiranyl, oxetanyl, tetrahydrofuryl, dioxolyl, tetrahydropyranyl, dioxanyl, oxazetidinyl, isoxazolidinyl, oxazolidinyl, morpholinyl, oxazinanyl, cyclopropyl, cyclobutyl, cyclopentyl, or cyclohexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by one or more substituents selected from —OR³⁴, —SR³⁴, —C(═O)NR³⁴R³⁵, —NR³⁴R³⁵, —NR³⁴C(═O)R³⁵, or —NR³⁴SO₂R³⁶, and wherein said aziridinyl, azetidinyl, pyrrolidinyl, pyrazolidinyl, imidazolidinyl, piperidinyl, piperazinyl, oxiranyl, oxetanyl, tetrahydrofuryl, dioxolyl, tetrahydropyranyl, dioxanyl, oxazetidinyl, isoxazolidinyl, oxazolidinyl, morpholinyl, oxazinanyl, cyclopropyl, cyclobutyl, cyclopentyl, or cyclohexyl may be substituted by one or more substituents selected from R², R³, R⁴, R⁵, R¹⁰, R¹¹, and R²¹;
W is phenyl-(C₁₋₆ alkylenyl), pyridyl-(C₁₋₆ alkylenyl), or indolyl-(C₁₋₆ alkylenyl), wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene, and wherein said phenyl, pyridyl, or indolyl is substituted by one or more groups selected from R³⁰ and R³¹;
Y is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein Y may be substituted by OH or by one or more substituents selected from F, Cl, and Br;
L is triazolyl, tetrazolyl, or oxadiazolyl;
R⁴ is H, —(C₁₋₆ alkylene)R⁶, —C(═O)R⁹, or —SO₂R¹², wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene, and wherein said phenyl, pyridyl, or indolyl is substituted by one or more groups selected from R³⁰ and R³¹;
R⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl, may be substituted by one or more R²⁶ substituents;
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —NHR²⁴, or —OR²⁵, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH or by one or more substituents selected from F, Cl, and Br;
R⁹ is —(C₁₋₆ alkylene)R²⁸, —NHR²⁴, or —OR²⁵;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be substituted by one or more substituents selected from CN, —OR²³, —SO₂R³⁷, —NR⁸R³³, —NR²⁴C(═O)R²³, and —NR²⁴SO₂R²³, provided that any one carbon atom of R¹² is not substituted by more than one CN or more than one —OR²³;
R^{b}, R^{c}, R⁸, R²⁰, R²², R²⁴, R²⁵, R³³, R³⁴, and R³⁵ are independently H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, 1-hexyl, may be substituted by OH;
R²⁶ is H, OH, F, Cl, Br, NH₂, or SH;
R²⁹ is H, —C(═O)CH₃, —C(═O)CH₂CH₃, —C(═O)(CH₂)₂CH₃, —C(═O)CH(CH₃)₂, —C(═O)(CH₂)₃CH₃, —C(═O)CH(CH₃)CH₂CH₃, —C(═O)CH₂CH(CH₃)₂, —C(═O)(CH₂)₄CH₃, —C(═O)CH₂CH(CH₃)CH₂CH₃, —C(═O)CH₂C(CH₃)₃, or —C(═O)(CH₂)₅CH₃;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —OCH₂CH₂OR²⁵;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² may be substituted with one, two, or three F;
R³⁶ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁷ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, 1-hexyl, may be substituted by OH;
or a pharmaceutically-acceptable salt thereof.

55. A compound according to claim 44 wherein Y is methyl.

56. A compound according to claim 1 having Formula IV:

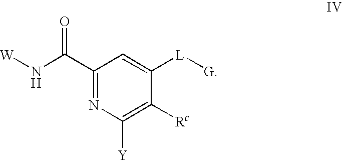

57. A compound according to claim 56 having Formula IVA:

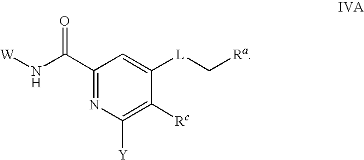

58. A compound according to claim 57 wherein L is triazolyl, tetrazolyl, or oxadiazolyl.

59. A compound according to claim 58 wherein L is

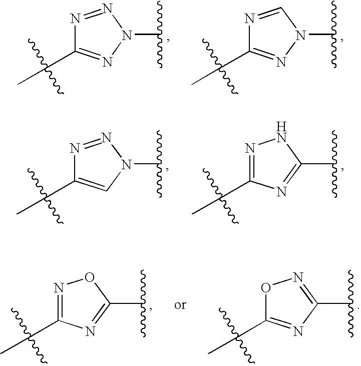

60. A compound according to claim 59 wherein L is

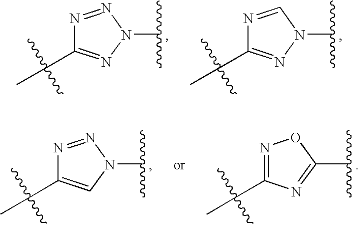

61. A compound according to claim 59 of Formula IVA-1

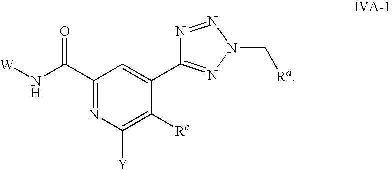

62. A compound according to claim 59 of Formula IVA-2

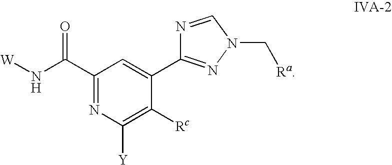

63. A compound according to claim 59 of Formula IVA-3

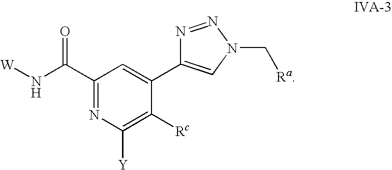

64. A compound according to claim 59 of Formula IVA-4

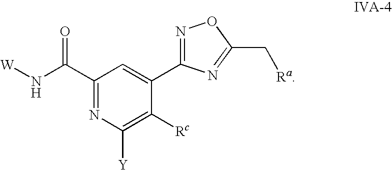

65. A compound according to any one of claims claim 60 wherein R^{a} is C₁₋₆ alkyl, 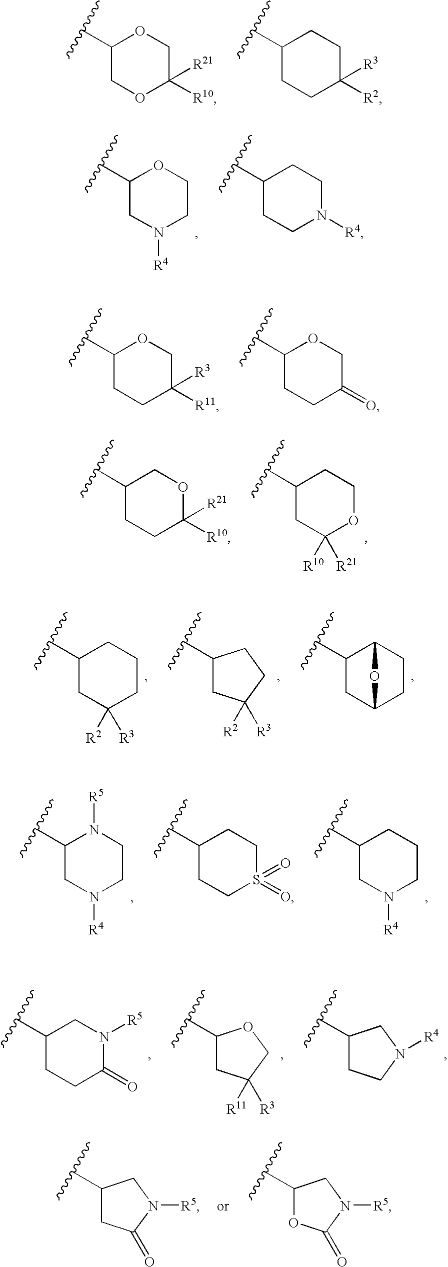
wherein said alkyl may be substituted by one or more substituents selected from ═OR³⁴, —SR³⁴, —C(═O)NR³⁴R³⁵, —NR³⁴R³⁵, —NR³⁴C(═O)R³⁵, or —NR³⁴SO₂R³⁵.

66. A compound according to claim 65 wherein R^{a} is C₁₋₆ alkyl, 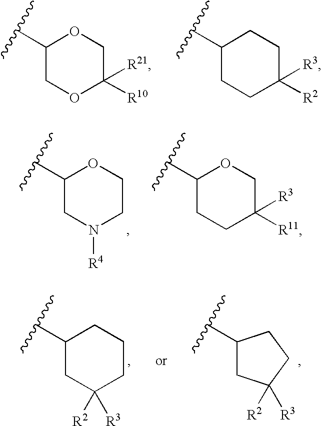
wherein said alkyl may be substituted by one or more substituents selected from —OR³⁴, —SR³⁴, —C(═O)NR³⁴R³⁵, —NR³⁴R³⁵, —NR³⁴C(═O)R³⁵, or —NR³⁴SO₂R³⁵.

67. A compound according to claim 65 wherein R^{a} is C₁₋₆ alkyl, wherein said alkyl may be substituted by one or more substituents selected from ═OR³⁴, —SR³⁴, —C(═O)NR³⁴R³⁵, —NR³⁴R³⁵, —NR³⁴C(═O)R³⁵, or —NR³⁴SO₂R³⁵.

68. A compound according to claim 67 wherein R^{a} is C₁₋₆ alkyl, wherein said alkyl may be substituted by one or more —OR³⁴;

69. A compound according to claim 66 wherein R^{a} is

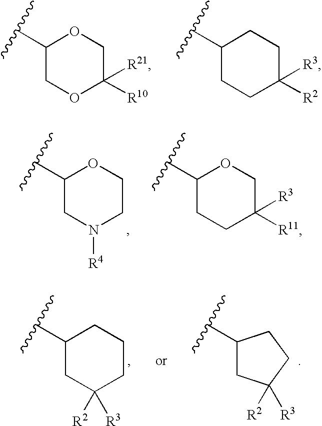

70. A compound according to claim 69 wherein R^{a} is 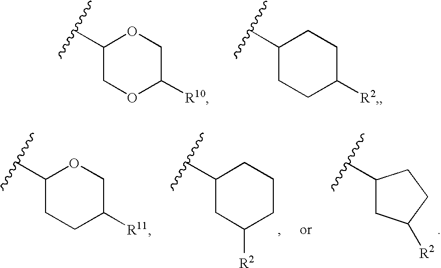
R² is CH(CH₃)OH, C(CH₃)₂OH, —NR⁸C(═O)(C₁₋₆ alkylene)R²⁸, or —NR⁸SO₂R³⁶;
R¹⁰ is CH(CH₃)OH, or C(CH₃)₂OH; and
R¹¹ is CH(CH₃)OH, C(CH₃)₂OH, —NR⁸C(═O)(C₁₋₆ alkylene)R²⁸, or —NR⁸SO₂R³⁶.

71. A compound according to claim 60 wherein:
R^{a} is methyl, ethyl, 1-propyl, 2-propyl, 1-butyl, 2-butyl, 2,2-dimethylethyl, 1-pentyl, 2-pentyl, 2,2-dimethylpropyl, 1-hexyl, aziridinyl, azetidinyl, pyrrolidinyl, pyrazolidinyl, imidazolidinyl, piperidinyl, piperazinyl, oxiranyl, oxetanyl, tetrahydrofuryl, dioxolyl, tetrahydropyranyl, dioxanyl, oxazetidinyl, isoxazolidinyl, oxazolidinyl, morpholinyl, oxazinanyl, cyclopropyl, cyclobutyl, cyclopentyl, or cyclohexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by one or more substituents selected from ═OR³⁴, —SR³⁴, —C(═O)NR³⁴R³⁵, —NR³⁴R³⁵, —NR³⁴C(═O)R³⁵, or —NR³⁴SO₂R³⁶, and wherein said aziridinyl, azetidinyl, pyrrolidinyl, pyrazolidinyl, imidazolidinyl, piperidinyl, piperazinyl, oxiranyl, oxetanyl, tetrahydrofuryl, dioxolyl, tetrahydropyranyl, dioxanyl, oxazetidinyl, isoxazolidinyl, oxazolidinyl, morpholinyl, oxazinanyl, cyclopropyl, cyclobutyl, cyclopentyl, or cyclohexyl may be substituted by one or more substituents selected from R², R³, R⁴, R⁵, R¹⁰, R¹¹, and R²¹;
W is phenyl-(C₁₋₆ alkylenyl), pyridyl-(C₁₋₆ alkylenyl), or indolyl-(C₁₋₆ alkylenyl), wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene, and wherein said phenyl, pyridyl, or indolyl is substituted by one or more groups selected from R³⁰ and R³¹;
Y is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein Y may be substituted by OH or by one or more substituents selected from F, Cl, and Br;
L is triazolyl, tetrazolyl, or oxadiazolyl;
R⁴ is H, —(C₁₋₆ alkylene)R⁶, —C(═O)R⁹, or —SO₂R¹², wherein said C₁₋₆ alkylene is methylene, ethylene, 1-propylene, 2-propylene, 1-butylene, 2-butylene, 2,2-dimethylethylene, 1-pentylene, 2-pentylene, 2,2-dimethylpropylene, or 1-hexylene, and wherein said phenyl, pyridyl, or indolyl is substituted by one or more groups selected from R³⁰ and R³¹;
R⁵ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl, may be substituted by one or more R²⁶ substituents;
R⁷ is methyl, ethyl, 1-propyl, 2-propyl, 1-butyl, 2-butyl, 2,2-dimethylethyl, 1-pentyl, 2-pentyl, 2,2-dimethylpropyl, 1-hexyl, —NHR²⁴, or —OR²⁵, wherein said methyl, ethyl, 1-propyl, 2-propyl, 1-butyl, 2-butyl, 2,2-dimethylethyl, 1-pentyl, 2-pentyl, 2,2-dimethylpropyl, or 1-hexyl may be substituted by OH or by one or more substituents selected from F, Cl, and Br;
R⁹ is —(C₁₋₆ alkylene)R²⁸, —NHR²⁴, or —OR²⁵;
R¹² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R¹² may be substituted by one or more substituents selected from CN, —OR²³, —SO₂R³⁷, —NR⁸R³³, —NR²⁴C(═O)R²³, and —NR²⁴SO₂R²³, provided that any one carbon atom of R¹² is not substituted by more than one CN or more than one —OR²³;
R^{b}, R^{c}, R⁸, R²⁰, R²², R²⁴, R²⁵, R³³, R³⁴, and R³⁵ are independently H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl;
R²³ is H, methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, 1-hexyl, may be substituted by OH;
R²⁶ is H, OH, F, Cl, Br, NH₂, or SH;
R²⁹ is H, —C(═O)CH₃, —C(═O)CH₂CH₃, —C(═O)(CH₂)₂CH₃, —C(═O)CH(CH₃)₂, —C(═O)(CH₂)₃CH₃, —C(═O)CH(CH₃)CH₂CH₃, —C(═O)CH₂CH(CH₃)₂, —C(═O)(CH₂)₄CH₃, —C(═O)CH₂CH(CH₃)CH₂CH₃, —C(═O)CH₂C(CH₃)₃, or —C(═O)(CH₂)₅CH₃;
R³¹ is Cl, Br, —OR³², methyl, ethyl, 1-propyl, 2-propyl, 1-butyl, 2-butyl, 2,2-dimethylethyl, 1-pentyl, 2-pentyl, 2,2-dimethylpropyl, 1-hexyl, or —OCH₂CH₂OR²⁵;
R³² is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein R³² may be substituted with one, two, or three F;
R³⁶ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl; and
R³⁷ is methyl, ethyl, 1-propyl, 2-propyl, 1-butyl, 2-butyl, 2,2-dimethylethyl, 1-pentyl, 2-pentyl, 2,2-dimethylpropyl, or 1-hexyl, wherein said methyl, ethyl, 1-propyl, 2-propyl, 1-butyl, 2-butyl, 2,2-dimethylethyl, 1-pentyl, 2-pentyl, 2,2-dimethylpropyl, 1-hexyl, may be substituted by OH;
or a pharmaceutically-acceptable salt thereof.

72. A compound according to claim 60 wherein Y is methyl.

73. A compound according to claim 60 wherein W is phenyl-(C₁₋₆ alkylenyl), wherein said phenyl is substituted by one or more groups selected from R³⁰ and R³¹.

74. A compound according to claim 73 wherein W is phenylmethyl, wherein said phenyl is substituted by one or more groups selected from R³⁰ and R³¹.

75-76. (canceled)

77. A compound having the structure:

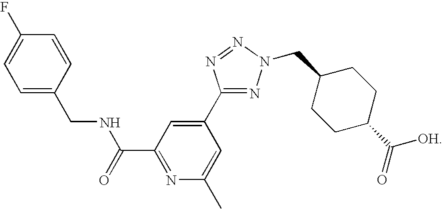

78. A compound having the structure:

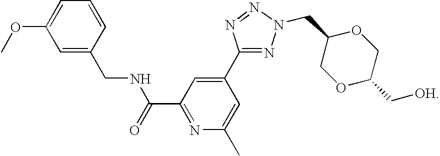

79. A compound having the structure:

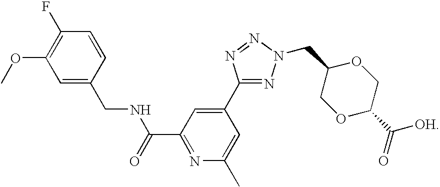

80. A compound having the structure:

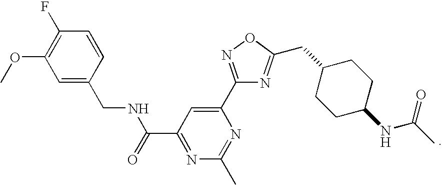

81. A compound having the structure:

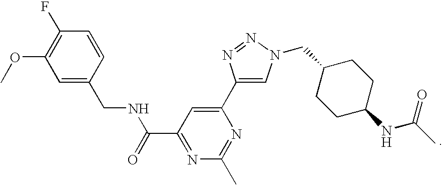

82. A pharmaceutical composition, comprising a compound according to claim 1, or a pharmaceutically acceptable salt thereof, admixed with a pharmaceutically acceptable carrier, excipient, or diluent.

83. A method for inhibiting an MMP-13 enzyme in an animal, comprising administering to the animal an MMP-13 inhibiting amount of a compound according to claim 1, or a pharmaceutically acceptable salt thereof.

84. A method for treating a disease mediated by an MMP-13 enzyme, comprising administering to a patient suffering from such a disease a nontoxic effective amount of a compound according to claim 1, or a pharmaceutically acceptable salt thereof.

85. A method for treating arthritis, comprising administering to a patient suffering from an arthritis disease a nontoxic antiarthritic effective amount of a compound according to claim 1, or a pharmaceutically acceptable salt thereof.

86. A-method according to claim 85, wherein the arthritis is osteoarthritis.

87. A method according to claim 85 wherein the arthritis is rheumatoid arthritis.

88. A method according to claim 85 wherein the arthritis is psoriatic arthritis.

89-94. (canceled)
